(19) United States
(12) Patent Application Publication
(10) Pub. No.: US 2026/0146995 A1
(43) Pub. Date: May 28, 2026

(54) SEMI-QUANTITATIVE LATERAL FLOW DEVICE AND METHOD FOR DETECTING BIOMARKERS IN UNPROCESSED SALIVA

(71) Applicant: Sparked Inc., Toronto (CA)

(72) Inventors: Stephanie Buryk-Iggers, Toronto (CA); Michael Glogauer, Toronto (CA); Patrick Lawler, Westmount (CA); Morteza Jeyhani, Toronto (CA)

(21) Appl. No.: 19/394,825

(22) Filed: Nov. 19, 2025

Related U.S. Application Data
(60) Provisional application No. 63/723,839, filed on Nov. 22, 2024.

(51) Int. Cl. G01N 33/543; G01N 33/68

(52) U.S. Cl. CPC G01N 33/54388; G01N 33/6887; G01N 2333/4737; G01N 2333/5412; G01N 2333/575; G01N 2800/32; G01N 2800/50

(57) ABSTRACT

Provided herein are sensitive lateral flow devices (LFDs) configured for use in an assay to measure the levels of a cardiometabolic disease risk biomarker in a sample from a subject, such as a saliva sample. Also provided herein are methods of using the LFDs disclosed herein in assays for measuring the level of a cardiometabolic disease risk biomarker in a sample from a subject, such as a saliva sample. Further provided herein are methods of manufacturing the LFDs disclosed herein.

### RELATED APPLICATION

This application claims benefit of U.S. provisional patent application Ser. No. 63/723,839 filed Nov. 22, 2024, incorporated herein by reference in its entirety.

### FIELD

The present disclosure relates to the field of lateral flow devices, and in particular, lateral flow devices capable of determining an individual's risk of having a cardiometabolic disease by measuring levels of cardiometabolic disease risk biomarkers in saliva.

### BACKGROUND

Developing affordable, accurate, and timely methods for CVD screening is critical to addressing the increased global prevalence of cardiovascular disease (CVD) 1. Current diagnostic practices for CVD and systemic inflammation rely on subjective clinical assessments and serum biomarker analysis of biomarkers such as serum C-reactive protein (CRP—including its high—sensitivity assay), which is linked to systemic and vascular inflammation and heart disease⁵.

Recent studies conducted by several groups have researched the compatibility of salivary CRP with serum CRP.^{3,6,7} Foley et al. noted significant associations (r=0.8) between serum and salivary CRP levels.⁸ Labat et al. identified a positive and significant correlation between salivary and serum CRP levels, suggesting that saliva could be an alternative means for screening and detecting cardiovascular disease risk.⁹ Additionally, CRP and inflammatory biomarkers are correlated with many other diseases and conditions.

Paper-based analytical devices have emerged as a promising tool for detecting serum and salivary biomarkers.^{10,11,12} They are particularly effective as point of care devices due to their affordability, accessibility and user-friendliness^{13,14,15}. Lateral flow devices (LFDs) and vertical flow devices (VFDs) are two arrangements of such paper-based analytical devices.

Park et al. (2019) demonstrated the detection of CRP in artificial samples within a clinically relevant range using a VFD, but was unable to achieve similarly sensitive detection with an LFD.¹⁶ However, interpretation of the VFD results required analysis of signal intensity, technical training and access to external devices such as a camera and image processing software,¹⁶ which limits the accessibility of the device and its suitability for use at point-of-care or at home.

Despite their affordability and ease of use, available LFD μPADs are not currently suitable for assessing low concentrations of a biomarker in samples such as saliva.

### SUMMARY

In accordance with an aspect of the present disclosure there is provided a semi-quantitative lateral flow device for measuring a cardiometabolic biomarker level in a saliva sample from a subject comprising:
I) a running liquid zone;
II) a sample pad zone configured to receive the sample;
III) a detection zone; and
IV an absorption zone;
wherein the running liquid zone, the sample pad zone, the detection zone, and the absorption zone are coupled sequentially by a continuous flow channel, and wherein the detection zone comprises at least one test dot configured to detect the cardiometabolic biomarker in saliva and at least one control dot.

In some embodiments, the cardiometabolic biomarker comprises C-reactive protein (CRP), brain natriuretic peptide (BNP) or interleukin 6 (IL6).

In some embodiments the detection zone comprises at least two test dots configured to detect the cardiometabolic biomarker in saliva.

In another embodiment the detection zone comprises three sequential test dots configured to detect the cardiometabolic biomarker in saliva.

In some embodiments, a test dot closest to the sample pad zone is configured to detect a low amount of the cardiometabolic biomarker in the sample, a middle test dot is configured to detect a moderate amount of the cardiometabolic biomarker in the sample, and/or a test dot furthest from the sample pad zone is configured to detect a high amount of the cardiometabolic biomarker in the sample.

In some embodiments, the cardiometabolic biomarker comprises CRP, and the low amount comprises 0.024 μg/mL CRP; the moderate amount comprises 0.048 μg/mL CRP; and/or the high amount comprises 0.072 μg/mL CRP.

In some embodiments, the cardiometabolic biomarker comprises BNP, and the low amount comprises about 5×10⁻⁷ μg/mL or less; the moderate amount comprises at least 5×10⁻⁷ μg/mL to less than 2×10⁻⁶ μg/mL; and/or the high amount comprises about 2×10⁻⁶ μg/mL or greater.

In some embodiments, the cardiometabolic biomarker comprises IL6, and wherein the low amount comprises about 5×10⁻⁷ μg/mL or less; the moderate amount comprises at least 5×10⁻⁷ μg/mL to less than 1.65×10⁻⁶ μg/mL; and/or the high amount comprises about 1.65×10⁻⁶ μg/mL or greater.

In some embodiments, each test dot is positioned along and coupled to the continuous flow channel.

In some embodiments, the sample pad zone, the detection zone, and the absorption zone and/or the running liquid zone are formed by a continuous nitrocellulose membrane.

In another embodiment, the nitrocellulose membrane has a pore size of 0.45 μm.

In yet another embodiment, the nitrocellulose membrane is coupled to a support layer by an adhesive layer.

In some embodiments, the support layer comprises aluminum foil.

In some embodiments, the at least one test dot comprises a capture agent.

In another embodiment, the capture agent comprises an anti-CRP antibody, an anti-BNP antibody or an anti-IL6 antibody.

In yet another embodiment, each of the at least one test dot comprises 0.2 μg of anti-CRP antibody, anti-BNP antibody or anti-IL6 antibody.

In some embodiments, the at least one control dot comprises a control capture agent.

In some embodiments, the LFD further measures one or more other cardiometabolic disease risk biomarker.

In some embodiments, the one or more other biomarkers of CVD comprises NT-proBNP.

In some embodiments, the device determines the risk that the subject has a cardiometabolic disease.

In some embodiments, the cardiometabolic disease comprises acute coronary syndrome, acute heart failure, myocardial infarction, stable atherosclerotic plaques, unstable angina, systemic hypertension, and/or cardiovascular mortality.

In some embodiments, the test dot has a width of about 1.5 mm.

In some embodiments, the device is for use in a colorimetric assay.

In another embodiment, the colorimetric assay is an immunoassay.

In another aspect, herein provided is a method of manufacturing an LFD disclosed herein, comprising:
I) mounting a nitrocellulose membrane to a support layer with an adhesive layer to form a body of the device;
II) cutting the nitrocellulose membrane with a laser cutter to provide a continuous flow channel, wherein the continuous flow channel comprises a running liquid zone, a sample pad zone, a detection zone, and an absorption zone;
III) adding at least one cardiometabolic biomarker capture agent to at least one test dot in the detection zone and adding at least one control capture agent to at least one control dot in the detection zone; and
IV) blocking the nitrocellulose membrane with a blocking agent.

In some embodiments, the method further comprises:
V) washing the nitrocellulose membrane with a washing solution.

In some embodiments, the washing solution comprises Tween 20.

In some embodiments, the blocking agent comprises skim milk.

In another aspect, herein provided is a method of determining a risk that a subject has a cardiometabolic disease, comprising:
I) combining a saliva sample from the subject with a cardiometabolic biomarker detection agent to form a test sample;
II) applying the test sample to a sample pad zone from an LFD herein disclosed;
III) applying a running liquid to a running liquid zone of the LFD in II); and
IV) applying a visualization agent to the detection zone;
   wherein the cardiometabolic biomarker detection agent and the visualization agent result in a visual change when combined, and wherein the visual change is indicative of the risk that the subject has cardiovascular disease.

In some embodiments, the cardiometabolic biomarker detection agent comprises a C-reactive protein (CRP) detection agent, a brain natriuretic peptide (BNP) detection agent or an interleukin 6 (IL6) detection agent.

In some embodiments, the cardiometabolic biomarker detection agent is an anti-CRP antibody conjugated to horseradish peroxidase, anti-BNP antibody conjugated to horseradish peroxidase or anti-IL6 antibody conjugated to horseradish peroxidase.

In some embodiments, the visualization comprises 3,3′,5,5′-Tetramethylbenzidine (TMB).

In some embodiments, the cardiometabolic biomarker detection agent binds a different cardiometabolic biomarker epitope than does the cardiometabolic biomarker capture agent in the LFD test dots.

In some embodiments, the running liquid comprises deionized water.

In some embodiments, a low amount of the cardiometabolic biomarker in the saliva is indicative that the subject has a low risk of cardiovascular disease; a moderate amount of the cardiometabolic biomarker in the saliva is indicative that the subject has a moderate risk of cardiovascular disease; or a high amount the cardiometabolic biomarker in the saliva is indicative that the subject has a moderate risk of cardiovascular disease.

In some embodiments, the cardiometabolic biomarker comprises CRP, and the low amount comprises 0.024 μg/mL CRP; the moderate amount comprises 0.048 μg/mL CRP; and/or the high amount comprises 0.072 μg/mL CRP.

In some embodiments, the cardiometabolic biomarker comprises BNP, and the low amount comprises about 5×10⁻⁷ μg/mL or less; the moderate amount comprises at least 5×10⁻⁷ μg/mL to less than 2×10⁻⁶ μg/mL; and/or the high amount comprises about 2×10⁻⁶ μg/mL or greater.

In some embodiments, the cardiometabolic biomarker comprises IL6, and the low amount comprises about 5×10⁻⁷ μg/mL or less; the moderate amount comprises at least 5×10⁻⁷ μg/mL to less than 1.65×10⁻⁶ μg/mL; and/or the high amount comprises about 1.65×10⁻⁶ μg/mL or greater

In another aspect, herein provided is a kit comprising an LFD herein disclosed, a running liquid, a solution comprising a CRP detection agent herein disclosed, and a solution comprising a visualization agent herein disclosed.

In some embodiments, the kit further comprises at least one receptable, at least one dropper, and/or instructions for use.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### BRIEF DESCRIPTION OF THE DRAWINGS

Further features and advantages of the disclosure will become apparent from the following detailed description taken in conjunction with the accompanying figures showing illustrative embodiments of the disclosure, which are now briefly described.

FIG. 1 shows a top view of an example embodiment of a lateral flow device 100.

FIG. 2 shows a perspective view of an example embodiment of the lateral flow device 100.

FIG. 3 shows a partially exploded view of a multi-layer LFD 100 in an example illustration of the disclosure, illustrating the layers of the LFD 100 and agents embedded in the LFD 100.

FIG. 4 shows an overview of an alternative embodiment of an LFD 100 for the detection and stratification readout of a cardiometabolic disease risk biomarker in an example illustration of the disclosure. Also shown is a schematic of an assay using the LFD 100.

FIG. 5 shows a repeating dispenser apparatus for dispensing microliters of fluid for use in the production of the LFD 100 in an example embodiment of the disclosure. The repeating dispenser is used to spot capture agent (e.g., anti-CRP capture antibodies) to the surface of detection zone of the LFD 100.

FIG. 6 shows another apparatus for dispensing microliters of fluid (Mahmud et al., 2020) such as the capture agent (e.g., anti-CRP capture antibodies) for use in the production of the LFD 100 in an example embodiment of the disclosure.

FIG. 7 shows the location of the detection agent 120, capture agent, and control capture agent 118 on the LFD 100 in an example illustration of the disclosure. The illustration further shows the schematic of bonding of the detection agent 120 to the target and the addition of TMB (an example visualization agent 121) to initiate a colorimetric reaction.

FIG. 8 shows results of an assay conducted with the LFD 100 across three trials using simulated samples comprising PSA and PBS buffer spiked with different amounts of human CRP in an example embodiment of the disclosure. As the concentration of hsCRP increases the number of test dots exhibiting a signal and the intensity of the signal across positive test dots increases.

FIG. 9 shows results of an assay conducted with the LFD 100 across a single trial using artificial saliva containing hsCRP in an example embodiment of the disclosure. As the concentration of hsCRP increases the number of test dots 114 exhibiting a signal and the intensity of the signal across positive test dots 114 increases.

FIG. 10 shows results of an assay conducted with the LFD 100 in human saliva from a patient with a positive CVD diagnosis (dilated cardiomyopathy) in an example embodiment of the disclosure. The patient had a known CRP serum concentration of 90 mg/mL translating to a CRP saliva concentration of 22.5 μg/mL (serum CRP concentration is typically 4000-fold higher than salivary CRP concentration).

FIG. 11A shows artificial samples with known concentrations of CRP (0.09 μg/mL, 0.006 μg/mL, 0.003 μg/mL, 0 μg/mL) run through the LFD using de-ionized water as the running liquid 122 both immediately after rinsing and 6 minutes after rinsing with TMB in an example embodiment of the disclosure.

FIG. 11B shows artificial samples with known concentrations of CRP (0.09 μg/mL, 0.006 μg/mL, 0.003 μg/mL, 0 μg/mL) run through the LFD using 2% BSA running liquid 122 both immediately after rinsing and 6 minutes after rinsing with TMB in an example embodiment of the disclosure.

FIG. 12A shows artificial samples with known concentrations of CRP (0.09 μg/mL, 0.06 μg/mL, 0.03 μg/mL, 0 g/mL) run through the LFD using 2% Tween in PBS both immediately after rinsing with TMB and 6 minutes after rinsing with TMB in an example embodiment of the disclosure.

FIG. 12B shows artificial samples with known concentrations of CRP (0.09 μg/mL, 0.06 μg/mL, 0.03 μg/mL, 0 μg/mL) run through the LFD using 2% Tween in BSA and PBS both immediately after rinsing with TMB and 6 minutes after rinsing with TMB in an example embodiment of the disclosure.

FIG. 13 shows a LFD 100 in an example embodiment of the disclosure. The letters correspond with measurements of the zones or channel of the device where a is about 7 mm, b is about 2 mm, c is about 3 mm, d is about 5 mm, e is about 8 mm, f is about 13 mm, g is about 3.5 mm, h is about 45 mm, j is about 3.5 mm and k is about 10 mm.

FIG. 14A shows artificial samples with known concentrations of BNP (5.0×10⁻⁷ μg/mL) run through the LFD using 2% Tween in PBS 3 minutes after rinsing with TMP in an example embodiment of the disclosure.

FIG. 14B shows artificial samples with known concentrations of BNP (2.0×10⁻⁶ μg/mL) run through the LFD using 2% Tween in PBS 3 minutes after rinsing with TMP in an example embodiment of the disclosure.

FIG. 14C shows artificial samples with known concentrations of BNP (5.0×10⁻⁷ μg/mL) run through the LFD using 2% Tween in PBS 6 minutes after rinsing with TMP in an example embodiment of the disclosure.

FIG. 14D shows artificial samples with known concentrations of BNP (2.0×10⁻⁶ μg/mL) run through the LFD using 2% Tween in PBS 6 minutes after rinsing with TMP in an example embodiment of the disclosure.

FIG. 15 shows a calibration curve generated using a serial dilution of CRP run through the LFD in an example embodiment of the disclosure.

FIGS. 16A-16J show artificial samples with known concentrations of CRP run through the LFD used to generate the calibration curve shown in FIG. 15. The known concentrations of CRP used were 0 g/ml (FIG. 16A), 0.0005 μg/ml (FIG. 16B), 0.0025 μg/ml (FIG. 16C), 0.01 μg/ml (FIG. 16D), 0.02 μg/ml (FIG. 16E), 0.03 μg/ml (FIG. 16F), 0.04 μg/ml (FIG. 16G), 0.05 μg/ml (FIG. 16H), 0.06 μg/ml (FIG. 16I), and 0.072 μg/ml (FIG. 16J).

FIG. 17A-17J show replicate artificial samples with known concentrations of CRP run through the LFD used to generate the calibration curve shown in FIG. 15. The known concentrations of CRP shown are 0 μg/ml (FIG. 17A), 0.0005 μg/ml (FIG. 17B), 0.0025 μg/ml (FIG. 17C), 0.01 μg/ml (FIG. 17D), 0.02 μg/ml (FIG. 17E), 0.03 μg/ml (FIG. 17F), 0.04 μg/ml (FIG. 17G), 0.05 μg/ml (FIG. 17H), 0.06 μg/ml (FIG. 17I), and 0.072 μg/ml (FIG. 17J).

FIG. 18 shows a limit of detection graph generated using known concentrations of CRP run through the LFD in an example embodiment of the disclosure.

FIGS. 19A-19F show artificial samples with known concentrations of CRP run through the LFT used to generate the limit of detection graph shown in FIG. 18. The known concentrations of CRP were 0.0005 g/ml (FIG. 19A), 0.001 μg/ml (FIG. 19B), 0.0015 μg/ml (FIG. 19C), 0.002 μg/ml (FIG. 19D), 0.0025 μg/ml (FIG. 19E), and 0.003 μg/ml (FIG. 19F).

FIGS. 20A-20F show replicates of artificial samples with known concentrations of CRP run through the LFT used to generate the limit of detection graph shown in FIG. 18. The known concentrations of CRP used were 0.0005 μg/ml (FIG. 20A), 0.001 μg/ml (FIG. 20B), 0.0015 μg/ml (FIG. 20C), 0.002 μg/ml (FIG. 20D), 0.0025 μg/ml (FIG. 20E), and 0.003 μg/ml (FIG. 20F).

FIGS. 21A-21K show representative artificial samples with no added CRP run through the LFT demonstrating the limit of the blank.

Further aspects and features of the example embodiments described herein will appear from the following description taken together with the accompanying drawings.

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

### I. Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least±5% of the modified term if this deviation would not negate the meaning of the word it modifies. The modifier “about” used in connection with a quantity is inclusive of the stated value and has the meaning dictated by the context (e.g., it includes the degree of error associated with measurement of the particular quantity). In addition, all ranges disclosed herein are inclusive of the endpoints and also any intermediate range points, whether explicitly stated or not, and the endpoints are independently combinable with each other.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as up to 1%, 2%, 5% or 10%, for example.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

As used herein in the specification and in the claims, the phrase “at least one”, in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example”. The word “or” is intended to include “and” unless the context clearly indicates otherwise.

The term “sample” or “test sample” as used herein refers to any material in which the presence, absence, or amount of a target analyte is unknown and can be determined in an assay. The sample can be from any source, for example, any biological (e.g. human or animal samples, including clinical samples), environmental (e.g. water, soil or air) or natural (e.g. plants) source, or from any manufactured or synthetic source (e.g. food or drinks). The sample can be comprised or is suspected of comprising one or more analytes. The sample can be a “biological sample” comprising cellular and non-cellular material, including, but not limited to, tissue samples, saliva, sputum, urine, blood, serum, other bodily fluids and/or secretions. In at least one embodiment, the sample comprises saliva, sputum, oropharyngeal and/or nasopharyngeal secretions. In at least one embodiment, the sample comprises sputum. In at least one embodiment, the test sample comprises a sputum sample.

The term “target”, “analyte”, “target analyte” or “biomarker” as used herein refer to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte can be either isolated from a natural source or synthetic. The analyte can be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “subject” as used herein includes all members of the animal kingdom including mammals such as a mouse, a rat, a dog and a human.

The term “cardiometabolic disease” as used herein refers to any disease or condition caused by a disruption or dysregulation of a cardiac or metabolic process, or any disease or condition that may result in a disruption or dysregulation of a cardiac or metabolic process, including as caused by or related to inflammatory molecules. For example, the cardiometabolic disease can comprise a “cardiovascular disease” or “CVD”, which is a general term referring to a condition of the heart or blood vessels that can, for example, be congenital or have an onset later in a subject's life cycle. CVD can comprise, for example, acute coronary syndromes, heart failure, atherosclerotic cardiovascular disease, heart rhythm disorders, systemic hypertension, valvular heart disease, and cardiovascular mortality. The cardiometabolic disease can also comprise metabolic diseases, such as type 2 diabetes, and their risk factors, such as obesity.

The term “limit of the blank” or “LoB” as used herein refers the highest apparent analyte concentration expected to be found when analyzing a sample containing a zero concentration of the analyte (a blank sample). It represents the maximum background noise of the assay and is the first step in defining analytical sensitivity. The term “limit of detection” or “LoD” as used herein refers to the lowest concentration of an analyte that can be reliably detected (reliably distinguished from the Limit of Blank, or background noise). It defines the true analytical sensitivity of the assay.

The term “calibration curve” as used herein refers to a plot of the known concentrations of the analyte against the signal response that the assay measures. It shows a reliable linear regression model that can be used to accurately determine the concentration of the unknown samples based on their measured signal.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either or both of those included limits are also included in the description.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either”, “one of”, “only one of”, or “exactly one of”.

It should also be understood that, in certain methods described herein that include more than one step or act, the order of the steps or acts of the method is not necessarily limited to the order in which the steps or acts of the method are recited unless the context indicates otherwise.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary.

### II. Semi-quantitative LFDs

In accordance with the teachings herein, the inventors describe a semi-quantitative lateral flow devices (LFDs) 100 capable of detecting salivary levels of at least one cardiometabolic disease risk biomarker 119 in the saliva of a user. To overcome the disadvantages of signal intensity measurements, counting-based test readout methods have recently been developed for use in an LFD 100 for a number of applications, but none had succeeded in quantifying cardiometabolic disease risk biomarkers at clinically relevant concentrations in saliva. The inventors here developed a simple-to-interpret “counting” based readout that can use a colorimetric signal to display to the user their relative risk of cardiometabolic disease in a semi-quantitative manner. The LFD 100 embodiments described herein do not require electronic components or additional equipment, making them especially suitable for point-of-care and at-home testing applications. The LFDs disclosed herein can be visually interpreted without a need for technical training.

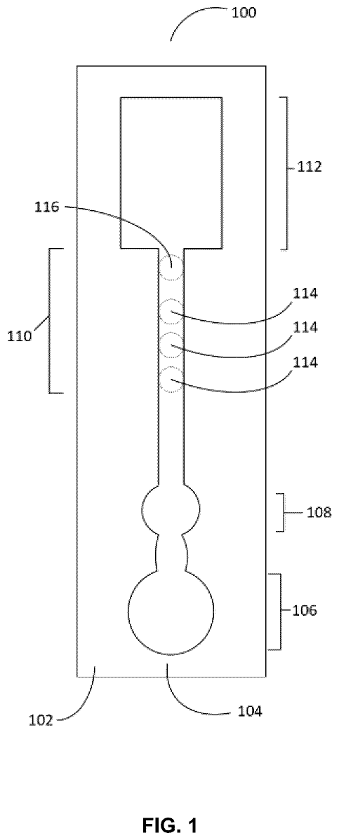

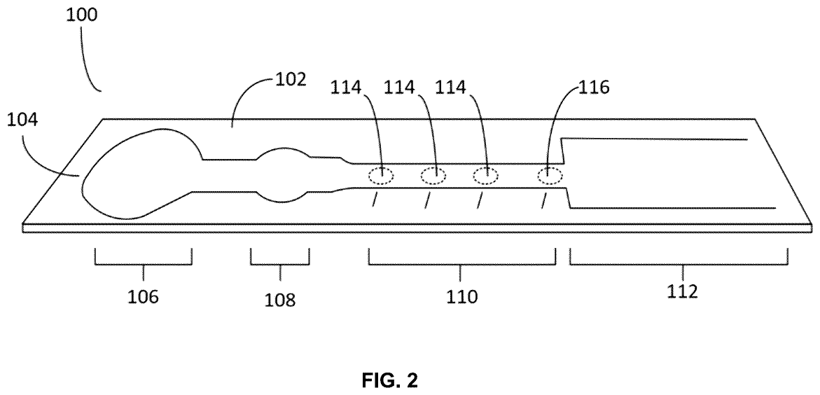

FIG. 1 and FIG. 2 provide an example embodiment comprising LFD 100 described herein. The LFD 100 comprises a porous layer 102, a continuous flow channel 104, a running liquid zone 106, a sample pad zone 108 downstream of the running liquid zone 106, a detection zone 110 downstream the sample pad zone 108 and the running liquid zone 106, wherein the detection zone 110 comprises three test dots 114 and a control dot 116, and an absorption zone 112 downstream of the detection zone 110, the sample pad zone 108, and the running liquid zone 106.

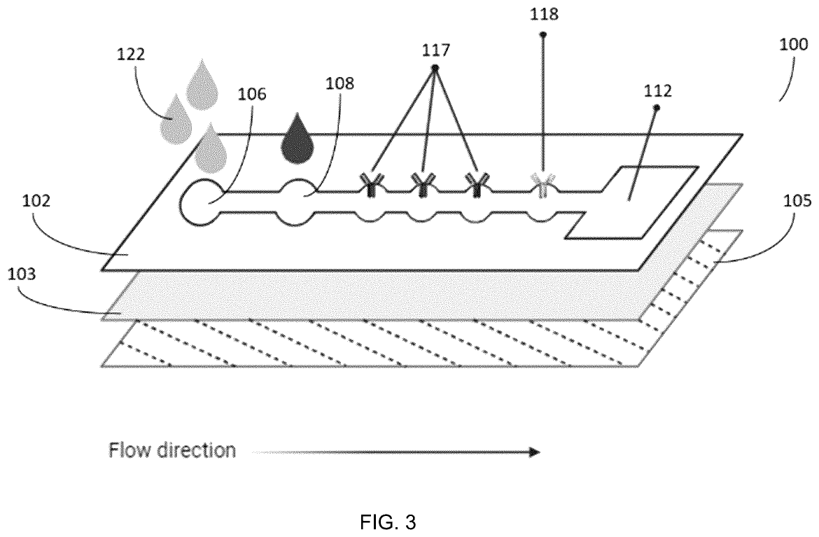

FIG. 3. Provides a partial exploded view of another example embodiment of LFD 100, comprising a porous layer 102, an adhesive layer 103, and a support layer 105. The LFD 100 also comprises a continuous flow channel 104, a running liquid zone 106, and a sample pad zone 108 downstream of the running liquid zone 106. A capture agent 117 for a cardiometabolic disease risk biomarker 119 is embedded in the test dots 114, and a control capture agent 118 is embedded into the control dot 116. Downstream of these zones in the absorption zone 112.

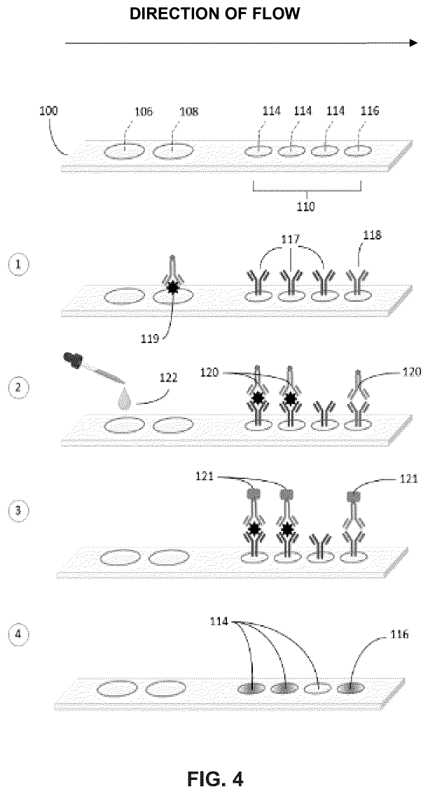

FIG. 4 illustrates yet another example embodiment of LFD 100 comprising a running liquid zone 106, a sample pad zone 108, and a detection zone 110 comprising three test dots 114 and a control dot 116. The test dots 114 are embedded with a capture agent 117 for a cardiometabolic disease risk biomarker 119, and the control dot 116 is embedded with a control capture agent 118. Steps 1 through 4 illustrate an example assay using the LDF 100. Step-1: A sample containing a cardiometabolic disease risk biomarker 119 to be quantified and a detection agent 120 for the cardiometabolic disease risk biomarker 119 is added to the sample pad zone 108. Step-2: Running liquid 122 is added to the running liquid zone 106 to transport the cardiometabolic disease risk biomarker 119 bound to the detection agent 120 to the detection zone 110, where the cardiometabolic disease risk biomarker 119 is also bound by the capture agent 117 in the test dots 114, and wherein the detection agent 120 is bound by the control capture agent 118 in the control dot 116. Step-3: Visualization agent 121 is added to the device to induce a visible change. Step-4: The test is read out by counting the number and/or intensity of test dots 114 in the detection zone 110 that have developed and confirming the presence of the developed control dot 116.

The LFD 100 can have a sensitivity capable of measuring salivary levels of a cardiometabolic disease risk biomarker 119, for example c-reactive protein (CRP), brain natriuretic peptide (BNP) and/or interleukin 6 (IL6). In some embodiments, the LFD 100 has a sensitivity capable of measuring clinically relevant levels of the cardiometabolic disease risk biomarker 119. Measuring serum levels of cardiometabolic disease risk biomarkers, such as CRP, BNP and/or IL6, is a known method of assessing an individual's risk of cardiometabolic disease; however, lower concentrations of cardiometabolic disease risk biomarkers in saliva (e.g., CRP is typically 200- to 500-fold lower in saliva than in serum) make measurement of salivary cardiometabolic disease risk biomarkers using LFDs 100 a challenge. Cardiometabolic disease risk biomarker levels (e.g., CRP, BNP and/or IL6 levels) can also be measured to assess an individual's systemic and/or acute inflammation levels and determine an individual's risk of developing diseases associated with inflammation. The LFD 100 described herein is, for example, capable of semi-quantitatively measuring salivary levels of cardiometabolic disease risk biomarkers in a saliva sample and communicating to a user their risk of developing a cardiometabolic disease such as CVD. The LFD 100 described herein can be used in a lateral flow assay (LFA) including direct, sandwich, competitive, and multiplex lateral flow assays. The LFD 100 can for example, include a counting-based readout, which can comprise at least three test dots 114 each capable of displaying a visual signal allowing a user to visually discern the results of an LFA using the LDF 100. The test dots 114 provide a semi-quantification of the user's salivary cardiometabolic disease risk biomarker level which can be indicative of the user's systemic or acute inflammation levels. Salivary cardiometabolic disease risk biomarker levels can also be interpreted to assess an individual's cardiometabolic disease risk, such as a risk of CVD (e.g., low, moderate, or high risk).

In some embodiments, the device can use a simple-to-interpret counting-based display comprising test dots 114 wherein e.g., a first test dot 114 corresponds with a low risk of the cardiometabolic disease, a second dot corresponds with moderate risk of the cardiometabolic disease and a third dot corresponds with a high risk of the cardiometabolic disease. For example, if testing a sample containing levels of a cardiometabolic disease risk biomarker associated with a low risk of the cardiometabolic disease, the first test dot 114 will display a visual signal; if testing a sample containing levels of a cardiometabolic disease risk biomarker associated with a moderate risk of the cardiometabolic disease, the first two test dots 114 will display a visual signal; and/or if testing a sample containing levels of a cardiometabolic disease risk biomarker 119 associated with a high risk of the cardiometabolic disease, all three test dots 114 will display a visual signal.

In some embodiments, the LFD 100 can further quantify the level of the cardiometabolic disease risk biomarker 119 in the sample based on the intensity of the visual signal at the test dots 114. The LFD 100 can measure cardiometabolic disease risk biomarker 119 levels in unprocessed saliva samples without the use of electronic components or additional equipment. The LFD 100 can be paper-based and can generate a visual signal that can be analyzed without the use of additional equipment. The LFD 100 described herein displays results to a user using a visual signal, for example, a colorimetric signal. The colorimetric signal can be produced by a colorimetric reporter substrate such as HRP. As such, the LFD 100 described herein is a cost-effective, portable, and simple to use making them ideal for simple and user-friendly point of care and/or home monitoring applications.

Accordingly, in an aspect, disclosed herein is a semi-quantitative LFD 100 for measuring levels of a cardiometabolic disease risk biomarker 119, in a sample from a subject comprising:
I) a sample pad zone 108 configured for receiving a sample;
II) a detection zone 110; and
III) an absorption zone 112;
   wherein the sample pad zone 108, the detection zone 110, and the absorption zone 112 are coupled sequentially by a continuous flow channel 104, and wherein the detection zone 110 comprises at least one test dot 114 configured to detect the cardiometabolic disease risk biomarker 119 in saliva and at least one control dot 116.

In another aspect, disclosed herein is a semi-quantitative LFD 100 for measuring levels of CRP in a sample from a subject comprising:
I) a sample pad zone 108 configured for receiving a sample;
II) a detection zone 110; and
III) an absorption zone 112;
   wherein the sample pad zone 108, the detection zone 110, and the absorption zone 112 are coupled sequentially by a continuous flow channel 104, and wherein the detection zone 110 comprises at least one test dot 114 configured to detect the CRP in saliva and at least one control dot 116.

In another aspect, disclosed herein is a semi-quantitative LFD 100 for measuring levels of BNP in a sample from a subject comprising:
I) a sample pad zone 108 configured for receiving a sample;
II) a detection zone 110; and
III) an absorption zone 112;
   wherein the sample pad zone 108, the detection zone 110, and the absorption zone 112 are coupled sequentially by a continuous flow channel 104, and wherein the detection zone 110 comprises at least one test dot 114 configured to detect the BNP in saliva and at least one control dot 116.

In another aspect, disclosed herein is a semi-quantitative LFD 100 for measuring levels of IL6 in a sample from a subject comprising:
I) a sample pad zone 108 configured for receiving a sample;
II) a detection zone 110; and
III) an absorption zone 112;
   wherein the sample pad zone 108, the detection zone 110, and the absorption zone 112 are coupled sequentially by a continuous flow channel 104, and wherein the detection zone 110 comprises at least one test dot 114 configured to detect the IL6 in saliva and at least one control dot 116.

In some embodiments, the device further comprises a running liquid zone 106, wherein the running liquid zone 106, the sample pad zone 108, the detection zone 110, and the absorption zone 112 are coupled sequentially by the continuous flow channel 104.

The term “lateral flow device” as used herein refers to a device that includes one or more fluid channels, flow channels, chambers, reservoirs or conduits that spontaneously drive a fluid across the device (e.g. by capillary force) and is capable of measuring the quantity or concentration of a target analyte in the fluid, for example a cardiometabolic disease risk biomarker 119 such as CRP, BNP and/or IL6. As used herein, a flow channel can include a continuous flow channel 104. An LFD 100 can include, for example, a microfluidic paper-based analytical device (μPAD) composed of a porous material, for example, nitrocellulose. As used herein LFD 100 can refer to a “μPAD LFD”. LFDs 100 are well known in the art and can be in a variety of formats designed by the person skilled in the art, including designs for use in direct, sandwich, competitive, and multiplex assays. An LFD 100 can also be used with a variety of detection agents 120, for example antibodies and aptamers conjugated to reporter substrates (see e.g., Sajid M, Kawde A and Daud M (2015) Design, Formats and Applications of Lateral Flow Assays, J Saudi Chem Soc., 19, 689-705, and Bahadir E B & Sezgintürk MK (2016) Lateral flow assays: Principles, designs and labels. Trends in Analytical Chemistry, 82, 286-306, the contents of which are incorporated by reference herein in their entireties).

The disclosed LFD 100 can include a porous layer 102, a continuous flow channel 104 and a plurality of different zones, such as a running liquid zone 106, a sample pad zone 108, a detection zone 110, and/or an absorption zone 112. The LFD 100 can use a detection agent 120, a capture agent 117, and a control capture agent 118, or a combination thereof to measure the amount of a target analyte, protein or biomarker in a sample. The LFD 100 can display semi-quantitative measurements through at least one test dot 114 and control dot 116 that can be present in the detection zone 110. The LFD 100 can further comprise a capture agent 117 embedded in the test dots 114 of the detection zone 110. The detection agent 120 can be added to the sample prior to the sample being applied to the sample pad zone 108 or included in the sample pad zone 108 as a lyophilized or dried reagent. Upon contact with the sample the detection agent 120 can specifically bind to a cardiometabolic disease risk biomarker 119 and can move along the continuous flow channel 104 of the LFD 100 through capillary action. The capture agent 117 embedded in the test dots 114 of the detection zone 110 can be specific to the cardiometabolic disease risk biomarker 119 and bind to the cardiometabolic disease risk biomarker 119 flowing into the detection zone 110 immobilizing it to the test dots 114, for example when the LFD 100 is used in an LFA.

The LFD 100 can be used in a sandwich style LFA utilizing a detection agent 120 such as an antibody specific to the cardiometabolic disease risk biomarker 119 conjugated to a reporter substrate. For example, the cardiometabolic disease risk biomarker 119 that is conjugated to the detection agent 120 can be captured by the embedded capture agent 117, while unbound detection agent 120 can flow through the detection zone 110 to a control dot 116 at the end of the detection zone 110. A control capture agent 118 can be embedded in the control dot 116 or included in the control dot 116 as a dried or lyophilized agent. The control dots 116 can serve as a positive control to ensure adequate flow of the sample across the length of the detection zone 110. The control dot 116 can also confirm the functionality of the detection agent 120 and a colorimetric visualization agent 121. The control capture agent 118 can be specific to the detection agent 120 but not specific the target biomarker, analyte, or substrate, for example CRP, BNP and/or IL6. Any detection agent 120 that is not bound to the cardiometabolic disease risk biomarker 119 can flow through the detection zone 110 to the control dot 116 where the detection agent 120 is immobilized to the control dot 116 by the specific binding of the control capture agent 118 to the detection agent 120. A colorimetric visualization agent 121 can be applied to the detection zone 110 for example after the sample has moved by capillary action through the detection zone 110. The colorimetric visualization agent 121 will induce the reporter substrate to produce a visual signal (e.g., colorimetric signal) in the test dots 114 positively containing a detectable amount of the cardiometabolic disease risk biomarker.

The user can discern their risk of cardiometabolic disease by observing the number of test dots 114 displaying a visual signal and the intensity of the signal in each test dot 114. The visual signal can be displayed on sequentially ordered test dots 114 each corresponding with a low, moderate, and high risk of cardiometabolic disease. For example, a first test dot 114, second test dot 114, and third test dot 114 of the detection zone 110 can each correspond with an increasing risk of cardiometabolic disease. If only the first test dot 114 displays a visual signal, then the result can be interpreted as a low risk of cardiometabolic disease. If both the first and second test dot 114 displays a visual signal, then the result can be interpreted as a moderate risk of cardiometabolic disease. If all three test dots 114 display a visual signal, then the result can be interpreted as a high risk of cardiometabolic disease. Accordingly, the LFD 100 described herein is intended for rapid detection of the presence or absence of at least one cardiometabolic disease risk biomarker, such as CRP, BNP and/or IL6, in a sample such as saliva, without the need for electronic components, or costly or sophisticated equipment.

The term “specifically binds”, as used herein, generally indicates that a molecule binds to a target molecule more readily, for example with higher affinity, than it would bind to an unrelated target.

The term “cardiometabolic disease risk biomarker” or “biomarker of cardiometabolic disease risk”, as used herein, refers to a substance that is associated with a biological state or a biological process of a cardiometabolic disease, such as a disease state or a diagnostic or prognostic indicator of a disease or disorder (e.g., an indicator identifying the likelihood of the existence or later development of a cardiometabolic disease). The presence or absence of a cardiometabolic disease risk biomarker 119, or the increase or decrease in the concentration of a cardiometabolic disease risk biomarker 119, may be associated with and/or be indicative of a risk or probability of the presence of a particular state or process in a subject. Cardiometabolic disease risk biomarkers 119 may include, but are not limited to, cells or cellular components (e.g., a viral cell, a bacterial cell, a fungal cell, a cancer cell, etc.), small molecules, lipids, carbohydrates, nucleic acids, peptides, proteins, enzymes, antigens and antibodies. A biomarker may be derived from an infectious agent, such as a bacterium, fungus or virus, or may be an endogenous molecule that is found in greater or lesser abundance in a subject suffering from a disease or disorder as compared to a healthy individual (e.g., an increase or decrease in expression of a gene or gene product). The cardiometabolic disease risk biomarker can, for example, comprise C-reactive protein (CRP), high sensitivity CRP (hsCRP), brain natriuretic peptide (BNP), the N-terminal fragment of the prohormone for BNP (NT-proBNP), and/or interleukin 6 (IL6).

In some embodiments, the cardiometabolic disease risk biomarker 119 is or comprises CRP, hsCRP, BNP, NT-proBNP, and/or IL6. In another embodiment, the cardiometabolic disease risk biomarker 119 is or comprises CRP and/or hsCRP. In another embodiment, the cardiometabolic disease risk biomarker 119 is or comprises BNP and/or NT-proBNP. In another embodiment, the cardiometabolic disease risk biomarker 119 is or comprises CRP. In another embodiment, the cardiometabolic disease risk biomarker 119 is or comprises hsCRP. In another embodiment, the cardiometabolic disease risk biomarker 119 is or comprises BNP. In yet another embodiment, the cardiometabolic disease risk biomarker 119 is or comprises NT-proBNP. In a further embodiment, the cardiometabolic disease risk biomarker 119 is or comprises IL6.

As used herein, the term “C-reactive protein” or “CRP” refers to a phylogenetically highly conserved protein that participates in the systemic response to inflammation. It is an acute phase reactant which is upregulated in response to systemic inflammation and typically exposed during cell death or found on the surface of pathogens. CRP can be from any organism, and optionally as defined by GenBank Accession Number AAA52075. CRP can also be present in fluid samples from a subject, including saliva and serum.

When a very small amount and/or very small change in CRP can be detected by a test or assay, this is commonly referred to as a high-sensitivity CRP (hsCRP or hs-CRP) test or assay. CRP at levels detected in high-sensitivity CRP assays can also be referred to as hsCRP. CRP can be characterized as a cardiometabolic disease risk biomarker 119 and is associated with a risk of diseases and conditions including but not limited to type 2 diabetes mellitus, acute infection, inflammation, and inflammation related to cancer. CVDs associated with increased CRP levels include, for example, acute coronary syndrome, acute heart failure, myocardial infarction, stable atherosclerotic plaques, unstable angina, systemic hypertension, and/or cardiovascular mortality.

As used herein, the term “brain natriuretic peptide” or “BNP” refers to a hormone secreted by cardiomyocytes. As used herein, the term “NT-proBNP” or “N-terminal pro b-type natriuretic peptide”, also called “BNPT” refers to the N-terminal fragment of the prohormone for brain natriuretic peptide (proBNP) that is then cleaved from BNP. The proBNP comprising NT-proBNP and BNP can be from any organism, and is optionally as defined in GenBank accession number P16860. BNP and NT-proBNP levels in serum are known to be correlated with risk of cardiometabolic disease, for example heart failure.

As used herein, the term “interleukin 6” or “IL6” refers to a molecule that can act as a pro-inflammatory cytokine or an anti-inflammatory myokine, depending on the context. IL6 can be from any organism and is optionally as defined in GenBank accession number NP_000591, NP_001305024, or NP_001358025. IL6 concentrations in serum are known to be correlated with a risk of a variety of cardiometabolic diseases.

The LFD 100 described herein can be used in a variety of assays, such as a qualitative assay. The LFD described herein can also be used in semi-quantitative lateral flow assays, for example the LFD 100 can be used in a direct, competitive, sandwich or multiplex lateral flow assay (LFA). In some embodiments, the LFD 100 is configured for use in a direct LFA. In some embodiments, the LFD 100 is configured for use in a competitive LFA. In another embodiment, the LDF 100 is configured for use in a sandwich LFA. In yet another embodiment, the LFD 100 is configured for use in a multiplex LFA.

In some embodiments, the device can be used in an assay that is colorimetric, fluorescent, magnetic, and/or enzymatic. In some embodiments, the LFD 100 can be used in an assay that is colorimetric. In another embodiment, the LFD 100 can be used in an assay that is fluorescent. In yet another embodiment, the LFD 100 can be used in an assay that is magnetic. In a further embodiment, the LFD 100 can be used in an assay that is enzymatic.

The colorimetric, fluorescent, magnetic, and/or enzymatic assay can comprise a pair of compounds or reagents (such as a reporter substrate and visualization agent 121), that when both present, result in the colorimetric-, fluorescent-, magnetic-, and/or enzymatic-based reporting to indicate the presence of the cardiometabolic disease risk biomarker, such as CRP, BNP and/or IL6. For example, the detection agent 120 can comprise horseradish peroxidase and the visualization agent 121 can comprise the colorimetric visualization agent 3,3′,5,5′-Tetramethylbenzidine (TMB), which results in a colored product when it is oxidized by horseradish peroxidase. Many such pairs of compounds or reagents are known.

The LFD 100 can be used in a LFA which comprises a traditional enzyme-linked immunosorbent assay such as a direct ELISA, an indirect ELISA, a sandwich ELISA, or a competitive ELISA. In some embodiments, the LDF 100 is used in a LFA comprising a direct ELISA. In another embodiment, the LDF 100 is used in a LFA comprising an indirect ELISA. In another embodiment, the LDF 100 is used in a LFA comprising a multiplex ELISA. In yet another embodiment, the LDF 100 is used in a LFA comprising a sandwich ELISA. In a further embodiment, the LDF 100 is used in a LFA comprising a competitive ELISA.

### Layers of the Device

The porous layer 102 can be composed of a porous material such as nitrocellulose. The porous layer 102 can be the topmost layer of the LFD 100 and can be coupled to a support layer 105 by an adhesive layer 103 in between the porous layer 102 and the support layer 105 (see FIG. 3). The support layer 105 can comprise, for example, aluminum foil. The LFD 100 can have distinct zones and at least one continuous flow channels 104. The porous layer 102 provides the surface upon which a sample and running liquid 122 is placed, and within which at least one continuous flow channel 104 and the different zones are formed. Several materials are suitable for making the LFD 100 described herein. In at least one embodiment, the porous layer 102 can comprise one or more porous materials, for example, nitrocellulose. In at least one embodiment, the porous layer 102 comprises a porous material with a pore size of about 0.45 μm.

The composition of the porous layer 102 can affect the sensitivity of the LFD 100. For example, variations in the pore size of the nitrocellulose membrane can affect the sensitivity of the LFD 100 for detecting CRP, BNP and/or IL6 in a saliva sample. A suitable material for use in the porous layer 102 can be, for example, a nitrocellulose membrane with a pore size of about 0.45 μm.

The skilled person also recognizes that many alternatives to nitrocellulose paper are possible, for example, any material that allows flow could work, such as cellulose, or any other material that supports capillary flow. Accordingly, in at least one embodiment, the LFD 100 comprises nitrocellulose paper, cellulose, or any material that supports capillary flow.

As used herein, the term “capillary action” or “capillarity” refers to the process by which a molecule is drawn across the porous substrate, against external forces such as the pull of gravity, due to forces such as surface tension and attraction between molecules.

The LFD 100 described herein can comprise a continuous flow channel 104 that is formed by removing material from the porous layer 102 to create a periphery of the continuous flow channel 104. In at least one embodiment, a continuous flow channel 104 of the LFD 100 described herein is formed by the removal of material from the porous layer 102. In at least one embodiment, a CO₂ laser is used to remove material from the porous layer 102 to form at least one continuous flow channel 104. For example, in at least one embodiment a CO₂ laser is used to remove material from a porous layer 102 to form an absorption zone 112, a sample pad zone 108, a detection zone 110 and an absorption zone 112 within or along the continuous flow channel 104. In at least one embodiment, the CO₂ laser is used to remove material to form a first flow channel 104 and at least one additional flow channel 104. In at least one embodiment, the CO₂ laser is used to remove material external to the continuous flow channel 104, to indicate the position of at least one test dot 114. In at least one embodiment, the CO₂ laser is used to remove material external to the continuous flow channel 104 to indicate the position of at least one control dot 116.

Other methods for creating flow channels 104 and/or detection zones 110 are known in the art, and include, for example, wax printing using an inkjet or solid ink printer, or photolithography. In some embodiments, the continuous flow channel 104 is created using wax printing with an inkjet or solid ink printer. In another embodiment, the continuous flow channel 104 is created using photolithography.

Many suitable adhesive layers would be readily apparent to one of skill in the art. In at least one embodiment, the adhesive layer 103 can comprise 3M™ Adhesive Transfer Tape 9775WL+ (3M ID: 7100308642). In another embodiment, the adhesive layer 103 can comprise 3M™ Adhesive Transfer Tape 467MP (Uline catalogue ID: S-10042). In at least one embodiment, the adhesive layer 103 can be selected based on its heat-resistant properties.

In at least one embodiment, the material for the support layer 105 can be selected based on its heat resistant and/or hydrophobic properties. In at least one embodiment, the material for the support layer 105 can be selected based on its heat resistant properties. In at least one embodiment, the material for the support layer 105 can be selected based on its hydrophobic properties.

Suitable materials for the support layer 105 include, for example, aluminum, glass, nonwoven heat-resistant plastic, dense foam, and/or silicon-based backings or plates. In some embodiments, the support layer 105 comprises aluminum, glass, nonwoven heat-resistant plastic, dense foam, and/or silicon-based backings or plates. In at least one embodiment, the support layer 105 is or comprises aluminum. In at least one embodiment, the support layer 105 is aluminum foil. In at least one embodiment, the support later is or comprises glass. In at least one embodiment, the support layer 105 is or comprises nonwoven heat-resistant plastic. In at least one embodiment, the support layer 105 is or comprises dense foam. In at least one embodiment, the support layer 105 is or comprises silicon-based backings or plates.

Methods for creating a hydrophobic barrier on a support layer are known to the person skilled in the art.

The LFD 100 described herein can comprise different zones along the flow channel 104. For example, a running liquid zone 106, a sample pad zone 108, a detection zone 110, and an absorption zone 112. In at least one embodiment, the sample pad zone 108 is configured to receive a sample. In at least one embodiment, the running liquid zone 106 is configured to receive a running liquid 122. In at least one embodiment, the LFD 100 can comprise a sample pad zone 108 and a detection zone 110. In at least one embodiment, the LFD 100 can comprise a sample pad zone 108 and a detection zone 110 ordered sequentially. In at least one embodiment, the LFD 100 comprises a running liquid zone 106, a sample pad zone 108, a detection zone 110 and an absorption zone 112. In at least one embodiment, the LFD 100 comprises a running liquid zone 106, a sample pad zone 108, a detection zone 110 and an absorption zone 112 ordered sequentially.

In at least one embodiment, the detection zone 110 comprises at least one capture agent 117 specific to CRP, hsCRP, BNP, NT-proBNP, or IL6. In another embodiment, the detection zone 110 comprises at least one capture agent 117 specific to CRP and/or hsCRP. In another embodiment, the detection zone 110 comprises at least one capture agent 117 specific to BNP or NT-proBNP. In another embodiment, the detection zone 110 comprises at least one capture agent 117 specific to CRP. In another embodiment, the detection zone 110 comprises at least one capture agent 117 specific to hsCRP. In another embodiment, the detection zone 110 comprises at least one capture agent 117 specific to BNP. In yet another embodiment, the detection zone 110 comprises at least one capture agent 117 specific to NT-proBNP. In a further embodiment, the detection zone 110 comprises at least one capture agent 117 specific to IL6.

There are a number of configurations for an LFD 100 known to the person skilled the art. For example, the LFD 100 can have more than one flow channel 104, for example, each with a distinct detection zone 110. For example, when the LFD 100 is configured to detect more than one cardiometabolic disease risk biomarker, each cardiometabolic disease risk biomarker 119 can be assessed in a separate detection zone 110, for example along a separate flow channel 104. Alternatively, when the LFD 100 is configured to detect more than one cardiometabolic disease risk biomarker, the LFD 100 can comprise a single continuous flow channel 104 with a single detection zone 110, wherein the detection zone 110 comprises separate test dots 114 and control dots 116 for the independent assessment of each cardiometabolic disease risk biomarker. In at least one embodiment, the LFD 100 comprises a first continuous flow channel 104 with a detection zone 110 comprising test dots 114 configured to detect a first cardiometabolic disease risk biomarker, and a second continuous flow channel 104 having a detection zone 110 comprising test dots 114 configured to detect a second cardiometabolic disease risk biomarker. In at least one embodiment, the detection zone 110 comprises a capture agent 117 for a first cardiometabolic disease risk biomarker 119 and at least one additional capture agent 117 for a second cardiometabolic disease risk biomarker. In at least one embodiment, the detection zone 110 comprises a capture agent 117 for CRP, hsCRP, BNP, NT-proBNP, or IL6 and at least one additional capture agent 117 for at least one other cardiometabolic disease risk biomarker 119. In at least one embodiment, the LFD 100 comprises a detection zone 110 comprising at least one test dot 114 configured to detect CRP, hsCRP, BNP, NT-proBNP, or IL6, and at least one additional detection zone 110 having test dots 114 configured to detect at least one other cardiometabolic disease risk biomarker 119.

The term “zone”, as used herein, refers to a defined area on the surface of a material (e.g., porous substrate). For example, a zone can be formed by removing material from a nitrocellulose membrane using a CO2 laser.

In at least one embodiment, the sample pad zone 108 is for applying a running buffer and a sample. In at least one embodiment, the sample pad zone 108 is for applying a running buffer, a sample, and a detection agent 120 for a cardiometabolic disease risk biomarker 119. In at least one embodiment, the sample pad zone 108 is for applying a running buffer, a sample, and a detection agent 120 for CRP. In at least one embodiment, the sample pad zone 108 is adapted for applying a running buffer, a sample, and a detection agent 120 for BNP. In at least one embodiment, the sample pad zone 108 is adapted for applying a running buffer, a sample, and a detection agent 120 for IL6. In at least one embodiment, the sample pad zone 108 is for applying a mixture comprising a sample and at least one detection agent 120 for a cardiometabolic disease risk biomarker 119, for example an anti-CRP antibody, an anti-BNP antibody and/or an anti-IL6 antibody coupled to a reporter substrate. In at least one embodiment, the sample pad zone 108 is connected through a flow channel 104 to a detection zone 110 for indicating the presence, absence, or a range of levels of a cardiometabolic disease risk biomarker 119, for example, CRP, BNP and/or IL6. In at least one embodiment, the sample pad zone 108 is connected through a flow channel to a detection zone 110 for indicating the presence, absence, or a range of levels of CRP. In at least one embodiment, the sample pad zone 108 is connected through a flow channel to a detection zone 110 for indicating the presence, absence, or a range of levels of BNP. In at least one embodiment, the sample pad zone 108 is connected through a flow channel to a detection zone 110 for indicating the presence, absence, or a range of levels of IL6.

In at least one embodiment, the mixture is left to incubate for a period of time prior to the application of the mixture to the sample pad zone 108 to allow the binding of the at least one detection agent 120 for a cardiometabolic disease risk biomarker 119 to at least one cardiometabolic disease risk biomarker 119 present in the sample. In at least one embodiment, the mixture can be applied directly to the sample pad zone 108 without first incubating the mixture. In at least one embodiment, the mixture comprising the sample and the at least one detection antibody is applied directly to the sample pad zone 108 prior to adding a running liquid 122 to the running liquid zone 106. In at least one embodiment, the running liquid 122 transports the sample and the at least one detection antibody to the detection zone 110 through capillary action.

In at least one embodiment, a running liquid 122 is applied to the running liquid zone 106 after application of the sample to the sample pad zone 108. In at least one embodiment, the running liquid 122 comprises Tween 20 in phosphate buffered saline (PBS). In at least one embodiment, the volume of running liquid 122 is about 12 μL. In at least one embodiment, the volume of running liquid 122 is between about 12 UL and about 16 μL. In some embodiments, the volume of running liquid 122 is about 12.0 μL. In some embodiments, the volume of running liquid 122 is about 12.5 μL. In some embodiments, the volume of running liquid 122 is about 13.0 μL. In some embodiments, the volume of running liquid 122 is about 13.5 μL. In some embodiments, the volume of running liquid 122 is about 14.0 μL. In some embodiments, the volume of running liquid 122 is about 14.5 μL. In some embodiments, the volume of running liquid 122 is about 15.0 μL. In some embodiments, the volume of running liquid 122 is about 15.5 μL. In some embodiments, the volume of running liquid 122 is about 16.0 μL. In some embodiments, the running liquid 122 is 2% (v/v) Tween 20 in PBS.

### Detection Zone

The term “detection zone” as used herein refers to a zone within the continuous flow channel 104 that comprises at least one test dot 114 comprising a capture agent 117 and at least one control dot 116 comprising a control capture agent 118. The detection zone 110 is located between the sample pad zone 108 of an LDF 100 and the absorption zone 112 of the LFD 100. The at least one test dot 114 that can be configured to detect a cardiometabolic disease risk biomarker 119 such as CRP, BNP and/or IL6. In at least one embodiment, the capture agent 117 or detection agent 120 is embedded in the test dot 114. In at least one embodiment, the detection agent 120 is added directly to the sample and the capture agent 117 is embedded in the test dots 114 wherein the detection agent 120 and the capture agent 117 are both specific to the cardiometabolic disease risk biomarker. In at least one embodiment, the detection zone 110 comprises at least one test dot 114. In at least one embodiment, the detection zone 110 comprises at least one control dot 116. In at least one embodiment, the detection zone 110 comprises at least one test dot 114 and at least one control dot 116. In at least one embodiment, the detection zone 110 comprises two test dots 114. In at least one embodiment, the detection zone 110 comprises three test dots 114. In at least one embodiment, the detection zone 110 comprises four test dots 114. In at least one embodiment, the detection zone 110 comprises five test dots 114. In at least one embodiment, the detection zone 110 comprises six test dots 114. In at least one embodiment, the detection zone 110 comprises six or more test dots 114.

In at least one embodiment, the detection zone 110 comprises a region for detecting low risk of the cardiometabolic disease, a region for detecting moderate risk of the cardiometabolic disease, a region for detecting high risk of the cardiometabolic disease, and a control region sequentially. In some embodiments, the detection zone 110 comprises a region for detecting low levels of systemic inflammation, a region for detecting moderate levels of systemic inflammation, a region for detecting high levels of systemic inflammation. In at least one embodiment, the test dots 114 are configured to detect CRP and are located within the region to detect low, moderate, and high risk of the cardiometabolic disease, optionally CVD. In at least one embodiment, the test dots 114 are configured to detect CRP and are located within the region to detect low, moderate, and high levels of systemic inflammation. In at least one embodiment, the test dots 114 are configured to detect BNP and are located within the region to detect low, moderate, and high risk of the cardiometabolic disease, optionally CVD. In at least one embodiment, the test dots 114 are configured to detect IL6 and are located within the region to detect low, moderate, and high risk of the cardiometabolic disease, optionally CVD. In at least one embodiment, the test dots 114 are configured to detect IL6 and are located within the region to detect low, moderate, and high levels of systemic inflammation. In at least one embodiment, the test dots 114 are configured to detect a cardiometabolic disease risk biomarker 119 and are located within the region to detect low, moderate, and high risk of the cardiometabolic disease. In at least one embodiment, the test dots 114 are configured to detect a cardiometabolic disease risk biomarker 119 and are located within the region to detect low, moderate, and high levels of systemic inflammation. In at least one embodiment, the region to detect low risk or low levels of systemic inflammation is closest to the sample pad zone 108, the region to detect moderate risk or moderate levels of systemic inflammation is downstream of the region to detect low risk or low levels of systemic inflammation and of the sample pad zone 108, and the region to detect high risk or high levels of systemic inflammation is downstream of the sample pad zone 108, the region to detect low risk or low levels of systemic inflammation, and the region to detect moderate risk or moderate levels of systemic inflammation. In at least one embodiment, the test dot 114 closest to the sample pad zone 108 is configured to detect a low amount of the cardiometabolic disease risk biomarker in the sample, a middle test dot 114 is configured to detect a moderate amount of the cardiometabolic disease risk biomarker 119 in the sample, and/or a test dot 114 furthest from the sample pad zone 108 is configured to detect a high amount of the cardiometabolic disease risk biomarker 119 in the sample.

In at least one embodiment, the LFD is configured to detect the cardiometabolic disease risk biomarker 119 in a range from about 0.001 μg/mL to about 1 μg/mL. In at least one embodiment, the LFD is configured to detect the cardiometabolic disease risk biomarker 119 in a range from about 0.0001 μg/mL to about 1 μg/mL. In some embodiments, the LFD is configured to detect the cardiometabolic disease risk biomarker in a range from about 0.0001 μg/mL to about 0.1 μg/mL. In some embodiments, the LFD is configured to detect the cardiometabolic disease risk biomarker in a range from about 5×10⁻⁷ μg/mL to about 2×10⁻⁶ μg/mL. In some embodiments, the LFD is configured to detect the cardiometabolic disease risk biomarker in a range from about 5×10⁻⁷ μg/mL to about 1.65×10⁻⁶ g/mL.

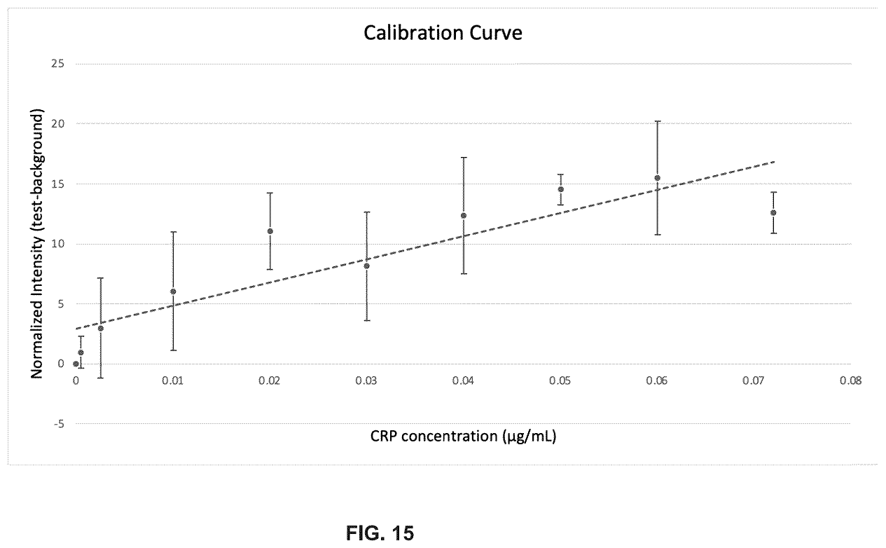

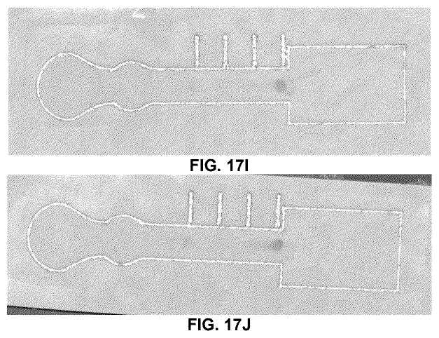

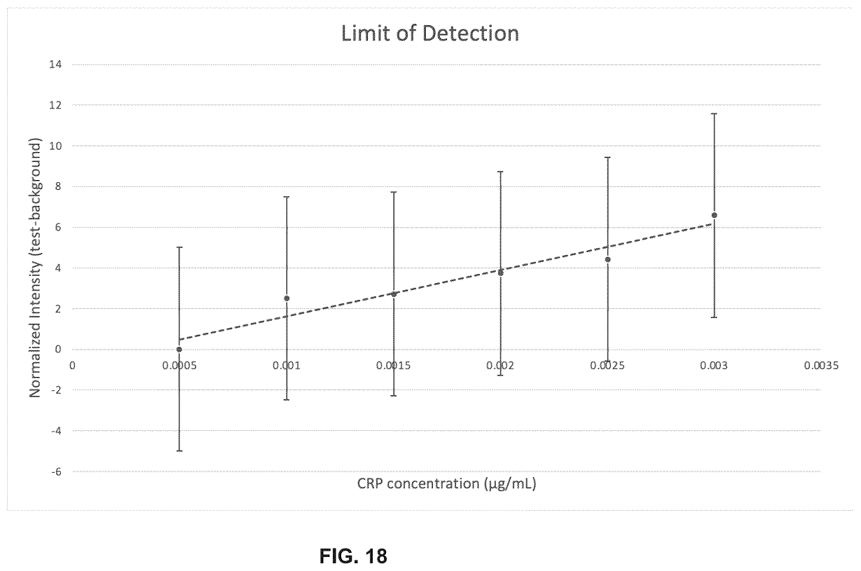

In some embodiments, the LFD is configured to detect CRP in a range from about 0.01 μg/mL to about 1 μg/mL. In another embodiment, the LFD is configured to detect CRP in a range from about 0.01 μg/mL to about 0.1 μg/mL. In another embodiment, the LFD is configured to detect CRP in a range from about 0.0001 μg/mL to about 0.1 μg/mL. In some embodiments, the LFD 100 has a sensitivity capable of measuring about 0.0005 μg/mL to about 0.0072 μg/mL of CRP (FIG. 15; FIG. 18).

In at least one embodiment, the low amount of CRP is about 0.024 μg/mL CRP. In at least one embodiment, the low amount of CRP is less than about 0.048 μg/mL. In at least one embodiment, the moderate amount of CRP is about 0.048 μg/mL. In at least one embodiment, the moderate amount is about 0.049-0.071 μg/mL CRP. In at least one embodiment, the moderate amount of CRP is about 0.049 μg/mL. In at least one embodiment, the moderate amount of CRP is about 0.050 μg/mL. In at least one embodiment, the moderate amount of CRP is about 0.051 μg/mL. In at least one embodiment, the moderate amount of CRP is about 0.052 μg/mL. In at least one embodiment, the moderate amount of CRP is about 0.053 μg/mL. In at least one embodiment, the moderate amount of CRP is about 0.054 μg/mL. In at least one embodiment, the moderate amount of CRP is about 0.055 μg/mL. In at least one embodiment, the moderate amount of CRP is about 0.056 μg/mL. In at least one embodiment, the moderate amount of CRP is about 0.057 μg/mL. In at least one embodiment, the moderate amount of CRP is about 0.058 μg/mL. In at least one embodiment, the moderate amount of CRP is about 0.059 μg/mL. In at least one embodiment, the moderate amount of CRP is about 0.060 μg/mL. In at least one embodiment, the moderate amount of CRP is about 0.061 μg/mL. In at least one embodiment, the moderate amount of CRP is about 0.062 μg/mL. In at least one embodiment, the moderate amount of CRP is about 0.063 μg/mL. In at least one embodiment, the moderate amount of CRP is about 0.064 μg/mL. In at least one embodiment, the moderate amount of CRP is about 0.065 μg/mL. In at least one embodiment, the moderate amount of CRP is about 0.066 μg/mL. In at least one embodiment, the moderate amount of CRP is about 0.067 μg/mL. In at least one embodiment, the moderate amount of CRP is about 0.068 μg/mL. In at least one embodiment, the moderate amount of CRP is about 0.069 μg/mL. In at least one embodiment, the moderate amount of CRP is about 0.070 μg/mL. In at least one embodiment, the moderate amount of CRP is about 0.071 μg/mL. In at least one embodiment, the high amount of CRP is about 0.072 μg/mL. In at least one embodiment, the high amount is greater than about 0.072 μg/mL.

In some embodiments, the LFD is configured to detect BNP in a range from about 5×10⁻⁷ μg/mL to about 2×10⁻⁶ μg/mL. In some embodiments, the LFD is configured to detect BNP in a range from less than 5×10⁻⁷ μg/mL to about 0.10 μg/mL.

In some embodiments, the low amount of BNP about 5×10^{-7.} μg/mL or less. In some embodiments, the low amount of BNP is about 5×10^{-7.} μg/mL. In some embodiments, the moderate amount of BNP is at least 5×10⁻⁷ μg/mL to less than 2×10⁻⁶ μg/mL. In some embodiments, the high amount of BNP is about 2×10⁻⁶ μg/mL or greater. In some embodiments, the high amount of BNP is about 2×10⁻⁶ μg/mL.

In some embodiments, the LFD is configured to detect IL6 in a range from about 5×10⁻⁷ μg/mL to about 1.65×10⁻⁶ μg/mL. In some embodiments, the LFD is configured to detect IL6 in a range from less than 5×10⁻⁷ μg/mL to at least 1.65×10⁻⁶ μg/mL. In some embodiments, the LFD is configured to detect IL6 in a range from less than 5×10⁻⁷ μg/mL to about 0.1 μg/mL.

In some embodiments, the low amount of IL6 is less than 5×10⁻⁷ μg/mL. In some embodiments, the low amount of IL6 is about 5×10⁻⁷ μg/mL. In some embodiments, the moderate amount of IL6 is at least 5×10⁻⁷ μg/mL to less than 1.65×10⁻⁶ μg/mL. In some embodiments, the high amount of IL6 is about 1.65×10⁻⁶ μg/mL or greater. In some embodiments, the high amount of IL6 is about 1.65×10⁻⁶ μg/mL.

In at least one embodiment, the test dots 114 are embedded with about 0.2 μL to about 0.6 μL of the capture agent. In at least one embodiment, the test dots 114 are embedded with about 0.2 μL of the capture agent. In at least one embodiment, the test dots 114 are embedded with about 0.3 L of the capture agent. In at least one embodiment, the test dots 114 are embedded with about 0.4 μL of the capture agent. In at least one embodiment, the test dots 114 are embedded with about 0.5 μL of the capture agent. In at least one embodiment, the test dots 114 are embedded with about 0.6 μL of the capture agent. In some embodiments, the capture agent 117 comprises unconjugated anti-CRP antibody. In some embodiments, the capture agent 117 comprises unconjugated anti-BNP antibody. In some embodiments, the capture agent 117 comprises unconjugated anti-IL6 antibody. In another embodiment, the capture agent 117 is at a concentration of about 1 mg/mL. In yet another embodiment, the unconjugated anti-CRP antibody is at a concentration of about 1 mg/mL. In yet another embodiment, the unconjugated anti-BNP antibody is at a concentration of about 1 mg/mL. In yet another embodiment, the unconjugated anti-IL6 antibody is at a concentration of about 1 mg/mL.

In at least one embodiment, at least one test dot 114 is configured to detect a cardiometabolic disease risk biomarker 119. In at least one embodiment, at least one test dot 114 is configured to detect CRP. In at least one embodiment, the detection zone 110 comprises three sequential test dots 114 configured to detect CRP. In at least one embodiment, the detection zone 110 comprises three sequential test dots 114 configured to detect CRP wherein a test dot 114 closest to the sample pad zone 108 is configured to detect a low amount of CRP in the sample, a middle test dot 114 is configured to detect a moderate amount of CRP in the sample, and/or a test dot 114 furthest from the sample pad zone 108 is configured to detect a high amount of CRP in the sample. In at least one embodiment, the test dots 114 are configured to detect CRP in saliva. In at least one embodiment, the detection zone 110 comprises two test dots 114 configured to detect CRP in saliva. In at least one embodiment, the detection zone 110 comprises three test dots 114 configured to detect CRP in saliva. In at least one embodiment, the detection zone 110 comprises four test dots 114 configured to detect CRP in saliva. In at least one embodiment, the detection zone 110 comprises five test dots 114 configured to detect CRP in saliva. In at least one embodiment, the test dots 114 are configured to detect a cardiometabolic disease risk biomarker 119 conjugated to a detection agent 120. In at least one embodiment, the test dots 114 are configured to detect CRP conjugated to a detection agent 120.

In at least one embodiment, at least one test dot 114 is configured to detect BNP. In at least one embodiment, the detection zone 110 comprises three sequential test dots 114 configured to detect BNP. In at least one embodiment, the detection zone 110 comprises three sequential test dots 114 configured to detect BNP wherein a test dot 114 closest to the sample pad zone 108 is configured to detect a low amount of BNP in the sample, a middle test dot 114 is configured to detect a moderate amount of BNP in the sample, and/or a test dot 114 furthest from the sample pad zone 108 is configured to detect a high amount of BNP in the sample. In at least one embodiment, the test dots 114 are configured to detect BNP in saliva. In at least one embodiment, the detection zone 110 comprises two test dots 114 configured to detect BNP in saliva. In at least one embodiment, the detection zone 110 comprises three test dots 114 configured to detect BNP in saliva. In at least one embodiment, the detection zone 110 comprises four test dots 114 configured to detect BNP in saliva. In at least one embodiment, the detection zone 110 comprises five test dots 114 configured to detect BNP in saliva. In at least one embodiment, the test dots 114 are configured to detect BNP conjugated to a detection agent 120.

In at least one embodiment, at least one test dot 114 is configured to detect IL6. In at least one embodiment, the detection zone 110 comprises three sequential test dots 114 configured to detect IL6. In at least one embodiment, the detection zone 110 comprises three sequential test dots 114 configured to detect IL6 wherein a test dot 114 closest to the sample pad zone 108 is configured to detect a low amount of IL6 in the sample, a middle test dot 114 is configured to detect a moderate amount of IL6 in the sample, and/or a test dot 114 furthest from the sample pad zone 108 is configured to detect a high amount of IL6 in the sample. In at least one embodiment, the test dots 114 are configured to detect IL6 in saliva. In at least one embodiment, the detection zone 110 comprises two test dots 114 configured to detect IL6 in saliva. In at least one embodiment, the detection zone 110 comprises three test dots 114 configured to detect IL6 in saliva. In at least one embodiment, the detection zone 110 comprises four test dots 114 configured to detect IL6 in saliva. In at least one embodiment, the detection zone 110 comprises five test dots 114 configured to detect IL6 in saliva. In at least one embodiment, the test dots 114 are configured to detect IL6 conjugated to a detection agent 120.

The test dots 114 can include a capture agent 117 that can specifically bind to a cardiometabolic disease risk biomarker 119 or to a complex between a detection agent 120 specific to a cardiometabolic disease risk biomarker 119 and the cardiometabolic disease risk biomarker 119 in a sample. Accordingly, the capture agent 117 can be a specific binding partner for a cardiometabolic disease risk biomarker 119 or a complex comprising the detection agent 120 and the cardiometabolic disease risk biomarker 119. In other words, the test dot 114 can detect the presence and/or the amount of the cardiometabolic disease risk biomarker 119 through e.g., a sandwich assay.

The control dots 116 include a control capture agent 118 that can specifically bind to the detection agent 120. Accordingly, the control capture agent 118 and the detection agent 120 can be a specific binding pair. Consequently, the control dot 116 can display a signal (once visualization agent 121 is applied) where detection agent 120 flows from the sample pad zone 108 passed the test dots 114, and to the control dot 116.

The detection agent 120 described herein can comprise an agent that selectively binds a cardiometabolic disease risk biomarker 119 (such as an antibody) conjugated to a reporter substrate, such as a colorimetric reporter substrate such as horseradish peroxidase (HRP). In at least one embodiment, the reporter substrate of the detection agent 120 comprises HRP. In at least one embodiment, the detection agent 120 comprises HRP conjugated to an anti-CRP antibody. In at least one embodiment, the detection agent 120 comprises HRP conjugated to an anti-BNP antibody. In at least one embodiment, the detection agent 120 comprises HRP conjugated to an anti-IL6 antibody In at least one embodiment, the reporter substrate can be colorimetric, fluorescent, magnetic, or enzymatic.

The term “configured to detect” as used herein refers to a configuration of one or more components of the LFD 100 (e.g. pore size, test dot 114 size, specific capture agent 117, amount or concentration of embedded capture agent 117 in the test dot 114, number of test dots 114, width of detection zone 110) that are arranged to either alone or in combination result in sufficient binding or immobilization of, for example, a cardiometabolic disease risk biomarker 119 or a detection agent 120 herein disclosed at a test dot 114 or control dot 116 to result in visual reporting of the cardiometabolic disease risk biomarker 119 or detection agent 120, when the LFD 100 is used in an assay with a sample from a subject. The one or more components of the LFD 100 can be configured to detect, for example, a clinically relevant concentration or range of concentrations of the cardiometabolic disease risk biomarker.

The capture agent 117 and control capture agent 118 can comprise a number of different molecules. For example, a capture agent 117 and a control capture agent 118 can each individually include a peptide, a protein, a carbohydrate, a lipid, a small molecule ligand, a nucleic acid, or a combination thereof. In at least one embodiment, the capture agent 117 and the control capture agent 118 each individually comprise a peptide or a protein. In at least one embodiment, the capture agent 117 and/or the control capture agent 118 comprises an antibody or an antigen binding fragment thereof.

In embodiments with a plurality of test dots 114, each test dot 114 can include the same capture agent. In some embodiments, the LFD 100 is configured to detect more than one cardiometabolic disease risk biomarker, wherein each cardiometabolic disease risk biomarker 119 is detected by a different capture agent, and wherein the different capture agents are embedded at a separate test dot 114 or set of test dots 114. For example, in some embodiments including a total of 4 test dots 114, all four test dots 114 can include the same capture agent, or, for example, 2 of the 4 test dots 114 can include a first capture agent 117 for a first cardiometabolic disease risk biomarker 119 and 2 test dots 114 can include a second capture agent 117 for a second cardiometabolic disease risk biomarker.

In at least one embodiment, the LFD 100 described herein can include a first flow channel 104 configured to detect CRP, BNP and/or IL6 and at least one additional flow channel 104 configured to detect a second cardiometabolic disease risk biomarker 119. In at least one embodiment, the LFD 100 comprises a first flow channel 104 comprising a detection zone 110 comprising a CRP specific capture agent 117, and a control capture agent 118 specific to a CRP specific detection agent 120. In at least one embodiment, the LFD 100 comprises a first flow channel 104 comprising a detection zone 110 comprising a BNP specific capture agent 117, and a control capture agent 118 specific to a BNP specific detection agent 120. In at least one embodiment, the LFD 100 comprises a first flow channel 104 comprising a detection zone 110 comprising a BNP specific capture agent 117, and a control capture agent 118 specific to a IL6 specific detection agent 120. The additional flow channel 104 can include a capture agent 117 specific to the second cardiometabolic disease risk biomarker 119 and a control capture agent 118 specific to a detection agent 120 specific to the second cardiometabolic disease risk biomarker 119. In at least one embodiment, the detection agent 120 comprises an antibody that specifically binds a cardiometabolic disease risk biomarker. In at least one embodiment, the detection agent 120 comprises an anti-CRP antibody, and optionally HRP. In at least one embodiment, the detection agent 120 comprises an anti-BNP antibody, and optionally HRP. In at least one embodiment, the detection agent 120 comprises an anti-IL6 antibody, and optionally HRP.

In at least one embodiment, the detection agent 120 comprises goat anti-CRP conjugated to HRP (Abcam catalogue no. ab19175). In at least one embodiment, the detection agent 120 comprises rabbit anti-BNP conjugated to HRP (LS BIO catalogue no. LS-C211190). In at least one embodiment, the detection agent 120 comprises rabbit anti-IL6 conjugated to HRP (Abcam catalogue no. ab106024).

In at least one embodiment, the capture agent 117 is an antibody that specifically binds the cardiometabolic disease risk biomarker. In at least one embodiment, the capture agent 117 is an anti-CRP antibody. In at least one embodiment, the capture agent 117 is an anti-BNP antibody. In at least one embodiment, the capture agent 117 is an anti-IL6 antibody.

In at least one embodiment, the capture agent 117 comprises goat anti-CRP (Sigma-Aldrich catalogue no. SAB4701017). In at least one embodiment, the capture agent 117 comprises mouse anti-BNP (Abcam catalogue no. ab20984). In at least one embodiment, the capture agent 117 comprises mouse anti-IL6 (Millipore Sigma catalogue nos. WH0003569M1, SAB1400140 or SAB5300275).

In at least one embodiment, the detection agent 120 binds a different epitope of the cardiometabolic disease risk biomarker 119 than does the capture agent 117 in the LFD 100 test dots 114.

In at least one embodiment, the control capture agent 118 is an antibody specific to the detection agent 120. In some embodiments, the control capture agent 118 is a secondary antibody. In at least one embodiment, the control capture agent 118 is an anti-goat antibody. In some embodiments, the control capture agent 118 is an anti-rabbit antibody.

In at least one embodiment, the control capture agent 118 comprises rabbit anti-goat (Abcam catalogue no. ab6697). In at least one embodiment, the control capture agent 118 comprises goat anti-rabbit (Abcam catalog no. ab6702).

The term “antibody” as used herein refers to monoclonal antibodies, polyclonal antibodies, chimeric antibodies, and humanized antibodies. The antibody can be from recombinant sources and/or produced in transgenic animals. The term “antigen binding fragment” or “antibody fragment” as used herein is intended to include without limitations Fv (a molecule comprising the V_{L} and V_{H}), single-chain variable fragment (scFv; a molecule comprising the V_{L} and V_{H} connected by a peptide linker), Fab, Fab′, F(ab′)2, dsFv, ds-scFv, single-domain antibodies (nanobodies), and multivalent presentations of these. Also included are dimers, minibodies, diabodies, and multimers thereof, bi-specific and multi-specific antibody fragments, and domain antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, sdAB, dimers, minibodies, diabodies, nanobodies, bispecific antibody fragments and other fragments can also be synthesized by recombinant techniques.

### Sample Pad Zone

The LFD 100 described herein can include a sample pad zone 108. The sample pad zone 108 can be configured to receive a sample. The sample once added to the sample pad zone 108 can travel through the flow channel 104 toward the detection zone 110 through capillary action. Accordingly, in at least one embodiment, the detection zone 110 is downstream from the sample pad zone 108. In at least one embodiment, the sample pad zone 108 includes stored dried reagents that can aid in the detection of a cardiometabolic disease risk biomarker 119. Example reagents on the sample pad zone 108 may include, but are not limited to, surfactants such as Triton X-100, Tween 20, or sodium dodecyl sulfate, etc.; polymers such as polyethylene glycol, poloxamer, polyvinylpyrrolidone (PVP), etc.; buffers such as phosphate-buffered saline, 4-(2-hydroxyethyl)-1-piperazineethanesulfonic acid (HEPES), Tris (hydroxymethyl) aminomethane (Tris), sodium borate, TRICINE, etc.; proteins such as albumin, etc.; enzymes such as protease, etc.; salts such as sodium chloride, sodium phosphate, sodium cholate, potassium phosphate, etc. The reagents can be applied to the sample pad by, e.g., soaking the material in the reagent solution, or through wicking the membrane via capillary flow. The treated sample pad can be dried by, e.g., air dry, heating at elevated temperatures, vacuum, or lyophilization. In at least one embodiment, the detection agent 120 can be present in the sample pad as a dried reagent (e.g. non-covalently bound).

### Detection Agent

A variety of detection agents 120 can be used in the LFD 100 described herein, including some described elsewhere in this disclosure. The specific detection agent 120 used can affect the detection sensitivity of the LFD 100. The amount of capture agent 117 spotted onto each test dot 114 can also affect the sensitivity of the LFD 100. The detection agent 120 can comprise an antibody specific to a cardiometabolic disease risk biomarker 119, for example CRP, BNP and/or IL6. In at least one embodiment, the detection agent 120 can comprise an antibody. In at least one embodiment, the detection agent 120 can comprise an anti-CRP antibody. In at least one embodiment, the detection agent 120 can comprise an anti-BNP antibody. In at least one embodiment, the detection agent 120 can comprise an anti-IL6 antibody. In at least one embodiment, the detection agent 120 comprises a reporter conjugated antibody. In at least one embodiment, the detection agent 120 comprises a reporter conjugated anti-CRP antibody. In at least one embodiment, the detection agent 120 comprises a reporter conjugated anti-BNP antibody. In at least one embodiment, the detection agent 120 comprises a reporter conjugated anti-IL6 antibody.

A variety of reporters (also referred to as “reporter substrates”) are appropriate for use in the LFD 100 described herein. The reporter substrate can be an enzyme (e.g., horse radish peroxidase, alkaline phosphatase and others commonly used in an ELISA) that can be visualized when combination with its specific visualization agent 121. In at least one embodiment, the visualization agent 121, when reacting with the reporter substrate, provides a colorimetric signal. In at least one embodiment, the reporter substrate is horse-radish peroxidase (HRP). In at least one embodiment, the reporter substrate is comprised in the detection agent 120. In at least one embodiment, the reporter substrate can be colorimetric, fluorescent, magnetic, or enzymatic. In at least one embodiment, the visualization agent 121 is colorimetric or fluorescent.

The LFD 100 described herein can include at least one detection agent 120, at least one capture agent 117, and at least one control capture agent 118. In at least one embodiment, the capture agent 117 is imbedded in the detection zone 110. In at least one embodiment, the capture agent 117 is imbedded in the test dots 114 of the detection zone 110 of the LFD 100 described herein. In at least one embodiment, the control capture agent 118 is embedded in the detection zone 110. In at least one embodiment, the control capture agent 118 is embedded in the control dot 116 of the detection zone 110. In at least one embodiment, the detection agent 120 comprises an anti-CRP antibody. In at least one embodiment, the capture agent 117 comprises an anti-CRP antibody. In at least one embodiment, the detection agent 120 comprises an anti-BNP antibody. In at least one embodiment, the capture agent 117 comprises an anti-BNP antibody. In at least one embodiment, the detection agent 120 comprises an anti-IL6 antibody. In at least one embodiment, the capture agent 117 comprises an anti-IL6 antibody. In at least one embodiment, the control capture agent 118 is an antibody specific to the selected detection agent 120.

In at least one embodiment, the capture agent 117 is an anti-CRP antibody. In at least one embodiment, the capture agent 117 is an anti-BNP antibody. In at least one embodiment, the capture agent 117 is an anti-IL6 antibody. In at least one embodiment, the capture agent 117 is a cardiometabolic disease risk biomarker 119 specific antibody. In at least one embodiment, the capture agent 117 is embedded in the detection zone 110. In at least one embodiment, the capture agent 117 is embedded in at least one test dot 114. In at least one embodiment, the capture agent 117 is embedded in a first test dot 114 and at least one additional capture agent 117 is embedded in a second test dot 114. In at least one embodiment, the control capture agent 118 is embedded in the detection zone 110. In at least one embodiment, the control capture agent 118 is embedded in a control dot 116.

### LFD Measurements

The test dots 114 and/or control dots 116 of the LFD 100 described herein can have a diameter sufficient to allow a user to visually discern the results of an assessment by visually counting the number of test dots 114 and/or control dots 116 displaying a signal, or based on the intensity of the signal at the test dots 114 and/or control dots 116. The diameter of the test dots 114 and/or control dots 116 can affect the sensitivity of the LFD 100 for measuring a cardiometabolic disease risk biomarker 119 in saliva samples. Without being bound by theory, the test dot 114 and control dot 116 can be narrower than the width of the detection zone 110 of continuous flow channel 104, and can be placed in a position that is, for example, equidistant from either edge of the width of the detection zone 110. For example, when the width of the detection zone 110 of the continuous flow channel 104 is about 2 mm, the test dot 114 and control dot 116 can have a diameter from about 1 mm to about 1.75 mm, optionally about 1.5 mm.

In some embodiments, the diameter of the test dot 114 and/or the control dot 116 is determined in relation to the width of the detection zone 110. In some embodiments, the width of the test dot 114 and/or the control dot 116 is about 50% to about 90% of the width of the detection zone 110 of the continuous flow channel 104. In another embodiments, the width of the test dot 114 and/or the control dot 116 is about 50% of the width of the detection zone 110 of the continuous flow channel 104. In another embodiments, the width of the test dot 114 and/or the control dot 116 is about 55% of the width of the detection zone 110 of the continuous flow channel 104. In another embodiments, the width of the test dot 114 and/or the control dot 116 is about 60% of the width of the detection zone 110 of the continuous flow channel 104. In another embodiments, the width of the test dot 114 and/or the control dot 116 is about 65% of the width of the detection zone 110 of the continuous flow channel 104. In another embodiments, the width of the test dot 114 and/or the control dot 116 is about 70% of the width of the detection zone 110 of the continuous flow channel 104. In another embodiments, the width of the test dot 114 and/or the control dot 116 is about 75% of the width of the detection zone 110 of the continuous flow channel 104. In another embodiments, the width of the test dot 114 and/or the control dot 116 is about 80% of the width of the detection zone 110 of the continuous flow channel 104. In another embodiments, the width of the test dot 114 and/or the control dot 116 is about 85% of the width of the detection zone 110 of the continuous flow channel 104. In another embodiments, the width of the test dot 114 and/or the control dot 116 is about 90% of the width of the detection zone 110 of the continuous flow channel 104.

In some embodiments, the test dots 114 have a diameter of about 1 mm to about 1.75 mm. In at least one embodiment, the test dots 114 have a diameter of about 1.4 mm to about 1.6 mm. In at least one embodiment, the test dots 114 have a diameter of about 1 mm. In at least one embodiment, the test dots 114 have a diameter of about 1.05 mm. In at least one embodiment, the test dots 114 have a diameter of about 1.1 mm. In at least one embodiment, the test dots 114 have a diameter of about 1.15 mm. In at least one embodiment, the test dots 114 have a diameter of about 1.2 mm. In at least one embodiment, the test dots 114 have a diameter of about 1.25 mm. In at least one embodiment, the test dots 114 have a diameter of about 1.3 mm. In at least one embodiment, the test dots 114 have a diameter of about 1.35 mm. In at least one embodiment, the test dots 114 have a diameter of about 1.4 mm. In at least one embodiment, the test dots 114 have a diameter of about 1.45 mm. In at least one embodiment, the test dots 114 have a diameter of 1.5 mm. In at least one embodiment, the test dots 114 have a diameter of about 1.55 mm. In at least one embodiment, the test dots 114 have a diameter of about 1.6 mm. In at least one embodiment, the test dots 114 have a diameter of about 1.65 mm. In at least one embodiment, the test dots 114 have a diameter of about 1.7 mm. In at least one embodiment, the test dots 114 have a diameter of about 1.75 mm.

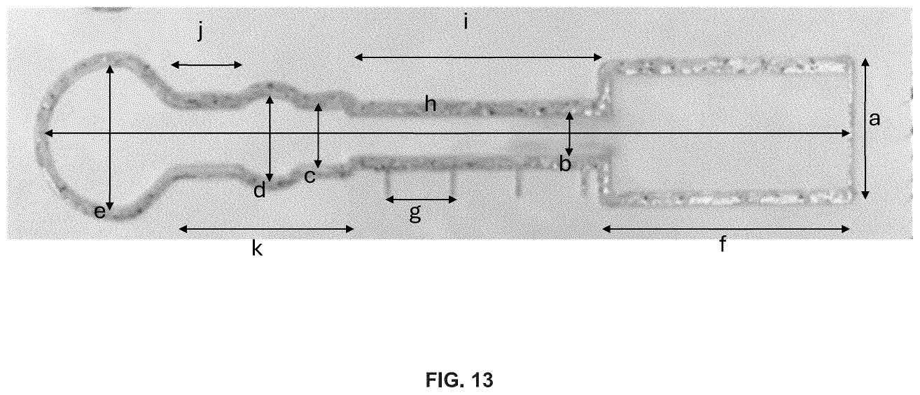

In at least one embodiment, the distance between the center of each test dot 114 is about 3.5 mm (e.g., FIG. 13). In at least one embodiment, the distance between the center of the last test dot 114 and the center of the control dot 116 is about 3.5 mm (e.g., FIG. 13),

In at least one embodiment, the length of the continuous flow channel 104 is about 45 mm (e.g., FIG. 13). In at least one embodiment, the length of the segment of the continuous flow channel 104 that lies between the running liquid zone 106 and sample pad zone 108 is about 3.5 mm (e.g., FIG. 13). In at least one embodiment, the length of the detection zone 110 is about 14 mm (e.g., FIG. 13). In at least one embodiment, the detection zone 110 is about 2 mm wide (e.g., FIG. 13).

In at least one embodiment, the running liquid zone 106 is about 8 mm wide (e.g., FIG. 13). In some embodiments, the absorption zone 112 is about 7 mm wide (e.g., FIG. 13). In some embodiments, the absorption zone 112 is about 13 mm in length (e.g., FIG. 13). In at least one embodiment, the LFD 100 disclosed herein is configured for use with about 12 μL of running liquid 122. In some embodiments, the LFD 100 is configured for use with 12 μL of running liquid for every 1 μL of detection agent-conjugated sample.

The LFD 100 disclosed herein can be scaled to different sizes, for example by preserving the proportionality between the different dimensions of features of the LFD 100, the volume of running liquid 122, and/or the volume and/or concentration of the embedded capture agent 117 and control capture agent 118. Alternatively, the LFD 100 disclosed herein can be scaled disproportionately, for example so long as the capillary flow rate, volume of running liquid 122, size of test dots 114, and concentration of capture agent 117 in the test dots 114 are configured to detect the cardiometabolic disease risk biomarker, optionally at predetermined clinically relevant concentrations.

### Cardiometabolic Diseases and Conditions, and Other Diseases and Conditions

Systemic and acute inflammation and a variety of cardiometabolic diseases can lead to increased CRP levels in the saliva of a human subject.^{25,26} For example diseases associated with systemic and acute inflammation that are correlated to CRP levels can include type 2 diabetes mellitus, acute infection, inflammation, and inflammation related to cancer. These cardiometabolic diseases include CVDs such as, acute coronary syndrome, acute heart failure, myocardial infarction, stable atherosclerotic plaques, unstable angina, systemic hypertension, and/or cardiovascular mortality.

A variety of cardiac stressors can lead to increased BNP and/or NT-proBNP levels in plasma, with correlated increases in the saliva of a human subject. For example, cardiac stressors that are associated with increases in BNP and/or NT-proBNP levels include decompensated or chronic heart failure, including cardiac stress or damage, pulmonary disorders, and renal impairment. Such cardiac stressors include, without limitation, cardiometabolic disorders such as acute and chronic heart failure, valvular disease, myocarditis, hypertrophy, arrhythmias, myocardial infarction, or pulmonary disorders such as pulmonary hypertension, embolism and/or chronic obstructive pulmonary disorder (COPD).

Cardiometabolic diseases associated with IL6 levels, include without limitation, atherosclerosis, coronary artery disease, hypertension, type 2 diabetes mellitus, metabolic syndrome, obesity, and heart failure.

In some embodiments, the LFD 100 described herein can determine the risk that a subject has a cardiometabolic disease. In at least one embodiment, the LFD 100 is configured to assess the risk of at least one of the following cardiometabolic diseases in a subject: acute coronary syndrome, acute heart failure, myocardial infarction, stable atherosclerotic plaques, unstable angina, systemic hypertension, or cardiovascular mortality or a combination thereof. In another embodiment, the LFD 100 is configured to assess the risk of acute coronary syndrome in a subject. In another embodiment, the LFD 100 is configured to assess the risk of acute heart failure in a subject. In another embodiment, the LFD 100 is configured to assess the risk of myocardial infarction in a subject. In another embodiment, the LFD 100 is configured to assess the risk of stable atherosclerotic plaques in a subject. In another embodiment, the LFD 100 is configured to assess the risk of unstable angina in a subject. In yet another embodiment, the LFD 100 is configured to assess the risk of systemic hypertension in a subject. In a further embodiment, the LFD 100 is configured to assess the risk of cardiovascular mortality in a subject.

In some embodiments, the LFD 100 described herein can determine the risk that a subject has a disease associated with acute or chronic inflammation. Accordingly, in at least one embodiment, the LFD 100 is configured to assess an individual's risk of type 2 diabetes mellitus, acute infection, inflammation, and inflammation related to cancer. In another embodiment, the LFD 100 is configured to assess the risk of type 2 diabetes mellitus in a subject. In another embodiment, the LFD 100 is configured to assess the risk of acute infection in a subject. In another embodiment, the LFD 100 is configured to assess the risk of inflammation in a subject. In yet another embodiment, the LFD 100 is configured to assess the risk of and inflammation related to cancer in a subject. In some embodiments, the LFD 100 is configured to assess an individual's level of systemic inflammation. In some embodiments, the LFD 100 is configured to assess an individual's level of acute inflammation.

In another embodiment, the LFD 100 described herein can determine the risk that a subject has a disease of condition associated with a change in concentration of a cardiometabolic disease risk biomarker disclosed herein, wherein the disease or condition is not a cardiometabolic disease or condition.

### III. Methods of Use

The LFDs 100 herein disclosed can be used in assays, for example lateral flow assays as described herein, to determine the risk that a subject has a disease associated with elevated CRP, BNP and/or IL6, or to determine the relative level of a cardiometabolic disease risk biomarker 119 in a sample.

Accordingly, in another aspect, disclosed herein is a method of determining a risk that a subject has a disease associated with elevated CRP, comprising:
I) combining a sample from the subject with a detection agent 120 herein disclosed to form a test sample, wherein the detection agent 120 is a CRP detection agent 120;
II) applying the test sample to a sample pad zone 108 of an LFD 100 herein disclosed;
III) applying a running liquid 122 herein disclosed to a running liquid zone 106 or the sample pad zone 108 of the LFD 100 in II); and
IV) applying a visualization agent 121 herein disclosed to the detection zone 110 of the LFD 100 in II);
   wherein the CRP detection agent 120 and the visualization agent 121 result in a visual change when combined, and where the visual change is indicative of the risk.

In a further aspect, disclosed herein is a method of determining a risk that a subject has a disease associated with elevated BNP, comprising:
I) combining a sample from the subject with a detection agent 120 herein disclosed to form a test sample, wherein the detection agent 120 is a BNP detection agent 120;
II) applying the test sample to a sample pad zone 108 of an LFD 100 herein disclosed;
III) applying a running liquid 122 herein disclosed to a running liquid zone 106 or the sample pad zone 108 of the LFD 100 in II); and
IV) applying a visualization agent 121 herein disclosed to the detection zone 110 of the LFD 100 in II);
   wherein the BNP detection agent 120 and the visualization agent 121 result in a visual change when combined, and where the visual change is indicative of the risk.

In a further aspect, disclosed herein is a method of determining a risk that a subject has a disease associated with elevated IL6, comprising:
I) combining a sample from the subject with a detection agent 120 herein disclosed to form a test sample, wherein the detection agent 120 is a BNP detection agent 120;
II) applying the test sample to a sample pad zone 108 of an LFD 100 herein disclosed;
III) applying a running liquid 122 herein disclosed to a running liquid zone 106 or the sample pad zone 108 of the LFD 100 in II); and
IV) applying a visualization agent 121 herein disclosed to the detection zone 110 of the LFD 100 in II);
   wherein the IL6 detection agent 120 and the visualization agent 121 result in a visual change when combined, and where the visual change is indicative of the risk.

In yet another aspect, disclosed herein is a method of measuring the relative level of a cardiometabolic disease risk biomarker 119 disclosed herein in a sample from a subject, comprising:
I) combining the sample from the subject with a detection agent 120 herein disclosed to form a test sample, wherein the detection agent 120 specifically binds to the cardiometabolic disease risk biomarker;
II) applying the test sample to a sample pad zone 108 of an LFD 100 herein disclosed;
III) applying a running liquid 122 herein disclosed to a running liquid zone 106 or the sample pad zone 108 of the LFD 100 in II); and
IV) applying a visualization agent 121 herein disclosed to the detection zone 110 of the LFD 100 in II);
   wherein the detection agent 120 and the visualization agent 121 result in a visual change when combined, and wherein the visual change is indicative of the relative level of the cardiometabolic disease risk biomarker.

Once the sample is combined with the detection agent 120, the solution can be left to incubate prior to being applied to the sample pad zone 108. In at least one embodiment, the sample and the detection agent 120 are added directly to the sample pad zone 108. In at least one embodiment, the sample and the detection agent 120 are incubated together for at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, or more prior to being added to the sample pad zone 108. In some embodiments, the sample and the detection agent 120 are incubated together for about 30 minutes to about 1 hour prior to being added to the sample pad zone 108. In another embodiment, the sample and the detection agent 120 are incubated together for about 10 minutes to about 1 hour prior to being added to the sample pad zone 108. In another embodiment, the sample and the detection agent 120 are incubated together for about 1 hour or more prior to being added to the sample pad zone 108. In yet another embodiment, the sample and the detection agent 120 are incubated together for at least 10 minutes prior to being added to the sample pad zone 108. In a further embodiment, the sample and the detection agent 120 are incubated together for at least 30 minutes prior to being added to the sample pad zone 108.

In another aspect, disclosed herein is a method of determining a risk that a subject has a disease associated with elevated CRP, comprising:
I) applying the sample to a sample pad zone 108 of an LFD 100 herein disclosed, wherein the sample pad zone 108 comprises a CRP detection agent 120 disclosed herein;
II) applying a running liquid 122 herein disclosed to a running liquid zone 106 or the sample pad zone 108 of the LFD 100 in I); and
III) applying a visualization agent 121 herein disclosed to the detection zone 110 of the LFD 100 in I);
   wherein the CRP detection agent 120 and the visualization agent 121 result in a visual change when combined, and wherein the visual change is indicative of the risk.

In another aspect, disclosed herein is a method of determining a risk that a subject has a disease associated with elevated BNP, comprising:
1) applying the sample to a sample pad zone 108 of an LFD 100 herein disclosed, wherein the sample pad zone 108 comprises a BNP detection agent 120 disclosed herein;
II) applying a running liquid 122 herein disclosed to a running liquid zone 106 or the sample pad zone 108 of the LFD 100 in I); and
III) applying a visualization agent 121 herein disclosed to the detection zone 110 of the LFD 100 in I);
   wherein the BNP detection agent 120 and the visualization agent 121 result in a visual change when combined, and wherein the visual change is indicative of the risk.

In another aspect, disclosed herein is a method of determining a risk that a subject has a disease associated with elevated IL6, comprising:
I) applying the sample to a sample pad zone 108 of an LFD 100 herein disclosed, wherein the sample pad zone 108 comprises a BNP detection agent 120 disclosed herein;
II) applying a running liquid 122 herein disclosed to a running liquid zone 106 or the sample pad zone 108 of the LFD 100 in I); and
III) applying a visualization agent 121 herein disclosed to the detection zone 110 of the LFD 100 in I);
   wherein the IL6 detection agent 120 and the visualization agent 121 result in a visual change when combined, and wherein the visual change is indicative of the risk.

In yet another aspect, disclosed herein is a method of measuring the relative level of a cardiometabolic disease risk biomarker 119 disclosed herein in a sample from a subject, comprising:
I) applying the sample to a sample pad zone 108 of an LFD 100 herein disclosed, wherein the sample pad zone 108 comprises a detection agent 120 herein disclosed that specifically binds to the cardiometabolic disease risk biomarker;
II) applying a running liquid 122 herein disclosed to a running liquid zone 106 or the sample pad zone 108 of the LFD 100 in I); and
III) applying a visualization agent 121 herein disclosed to the detection zone 110 of the LFD 100 in I);
   wherein the detection agent 120 and the visualization agent 121 result in a visual change when combined, and wherein the visual change is indicative of the relative level of the cardiometabolic disease risk biomarker.

A variety of different samples can be analyzed by the LFD 100 disclosed herein. In at least one embodiment, the sample comprises blood, plasma, serum, saliva, sputum, and/or urine. In at least one embodiment, the sample comprises blood. In at least one embodiment, the sample comprises plasma. In at least one embodiment, the sample comprises serum. In at least one embodiment, the sample comprises saliva. In at least one embodiment, the sample comprises sputum. In at least one embodiment, the sample comprises urine.

In some embodiments, the risk is low, moderate, or high as disclosed herein.

In some embodiments, the disease associated with elevated CRP is a CVD. In some embodiments, the CVD comprises acute coronary syndrome, acute heart failure, myocardial infarction, stable atherosclerotic plaques, unstable angina, systemic hypertension, and/or cardiovascular mortality. In another embodiment, the cardiovascular disease comprises acute coronary syndrome. In another embodiment, the CVD comprises acute heart failure. In another embodiment, the CVD comprises myocardial infarction. In another embodiment, the CVD comprises stable atherosclerotic plaques. In another embodiment, the CVD comprises unstable angina. In yet another embodiment, the CVD comprises systemic hypertension. In a further embodiment, the CVD comprises cardiovascular mortality.

In some embodiments, the disease associated with elevated CRP is a disease associated with chronic or acute inflammation. In another embodiment, the disease associated with elevated CRP is type 2 diabetes mellitus, acute infection, inflammation, and/or inflammation related to cancer. In another embodiment, the disease associated with elevated CRP is type 2 diabetes mellitus. In another embodiment, the disease associated with elevated CRP is acute infection. In yet another embodiment, the disease associated with elevated CRP is inflammation. In a further embodiment, the disease associated with elevated CRP is inflammation related to cancer.

In some embodiments, the disease associated with elevated BNP is decompensated or chronic heart failure. In some embodiments the disease associated with elevated BNP is cardiac stress or damage, heart failure, valvular disease, myocarditis, hypertrophy, arrhythmias, myocardial infarction, a pulmonary disorder, or renal impairment.

In some embodiments, the disease associated with elevated IL6 is atherosclerosis, coronary artery disease, hypertension, type 2 diabetes mellitus, metabolic syndrome, obesity, or heart failure.

In some embodiments, the method comprises comparing the level of the cardiometabolic disease risk biomarker 119, CRP, BNP, IL6 or antibody complex thereof to one or more controls or reference values. Controls may include, for example and without limitation, an expression level of the cardiometabolic disease risk biomarker 119 CRP, BNP or IL6 in a healthy patient or a reference expression level obtained or determined from samples of a group of healthy patients, which can be used to create a “control value”. A control value may be obtained from the historical expression data from a pool of healthy patients (e.g. a negative control value) or from a pool of patients with the disease (e.g. a positive control value). Controls may also include an internal control, for example the level of a cardiometabolic disease risk biomarker 119 CRP, BNP or IL6 relative to a second cardiometabolic disease risk biomarker 119 from the patient sample (e.g. the level of a cardiometabolic disease risk biomarker X 119 relative to the level of a cardiometabolic disease risk biomarker Y 119). Similarly, a reference value may include for example a threshold value, above or below which (depending on the specific cardiometabolic disease risk biomarker and reference value) may indicate the patient has a disease or increased risk thereof or may indicate the patient does not have a disease or has a decreased risk thereof.

Further description of the LFDs 100, porous layers 102, sample pad zones 108, detection zones 110, detection agents 120, test dots 114, control dots 116, capture agents 117, control capture agents 118, visualization agents 121, cardiometabolic disease risk biomarkers, diseases, biomarker levels, risk levels, and other components or features disclosed herein can also be applied to the methods of measuring the relative level of a cardiometabolic disease risk biomarker 119 and the methods of determining a risk that a subject has a disease associated with elevated CRP BNP and/or IL6 disclosed herein.

### IV. Methods of Manufacture

In another aspect, also provided is a method of manufacturing a LFD 100 described herein comprising:
I) mounting a porous layer 102 herein disclosed to a support layer 105 herein disclosed with an adhesive layer 103 herein disclosed to form a body of the LFD 100;
II) cutting the porous layer 102 to provide a continuous flow channel 104, wherein the continuous flow channel 104 comprises a running liquid zone 106, a sample pad zone 108, a detection zone 110, and an absorption zone 112;
III) adding at least one capture agent 117 herein disclosed to at least one test dot 114 in the detection zone 110 and adding at least one control capture agent 118 herein disclosed to at least one control dot 116 in the detection zone 110; and
IV) blocking the porous layer 102 with a blocking agent herein disclosed.

In some embodiments, the porous layer 102 is a nitrocellulose disclosed herein.

In another embodiment, the cutting in ii) comprises cutting with a laser.

In some embodiments, the capture agent 117 is a CRP capture agent. In some embodiments, the capture agent 117 is a BNP capture agent. In some embodiments, the capture agent 117 is a IL6 capture agent.

In at least one embodiment, the method of manufacturing further comprises V) washing the porous layer 102, optionally nitrocellulose membrane, with a washing solution. In at least one embodiment, the washing solution comprises Tween-20. In at least one embodiment, the blocking agent comprises skim milk. In at least one embodiment, the blocking agent comprises BSA. In some embodiments, the blocking agent comprises synthetic blockers, gelatin, or casein, or a combination thereof. In another embodiment, the blocking agent comprises synthetic blockers. In yet another embodiment, the blocking agent comprises gelatin. In a further embodiment, the blocking agent comprises casein.

Further, description of the devices, porous layers 102, sample pad zones 108, detection zones 110, detection agents 120, test dots 114, control dots 116, and other components or features disclosed herein can also be applied to the methods of manufacture disclosed herein.

### V. Kits

Also disclosed herein are kits that can be used for, e.g., measuring a user's risk of a cardiometabolic disease using the disclosed lateral flow devices 100. The kit can include an LFD 100 described herein, and one or more packages, receptacles, labels, or instructions for use. The kit can include at least one running liquid 122. The kit can include at least one buffer or reagent. The kit can also include other components to facilitate using the device and methods thereof. Examples of such components include, but are not limited to, one or more additional reagents, such as one or more dilution buffers; one or more reconstitution solutions; one or more wash buffers; one or more storage buffers, one or more control reagents, (one or more additional component(s), such as a sample collection device (e.g., a syringe, cotton swab, tongue depressor, and the like), and the like. Components (e.g., reagents, components, etc.) may also be provided in a form that is usable in a particular assay, or in a form that requires addition of one or more other components before use (e.g. in concentrate or lyophilized form). The buffers can be but are not limited to, phosphate buffered saline, sodium carbonate buffer, sodium bicarbonate buffer, borate buffer, Tris buffer, MOPS buffer, HEPES buffer, and combinations thereof. The choice of buffers and reagents will depend on the particular application, e.g., setting of the assay (point-of-care, research, clinical), analyte(s) to be assayed, the detection moiety used, etc. Such components may be provided individually or in combination(s) and may be provided in any suitable container such as a vial, a bottle, box, or a tube.

The kit may also include a packaging configured to contain the device and other components. The packaging may be a sealed packaging, such as a sterile sealed packaging. By “sterile” it is meant that there are substantially no microbes (such as fungi, bacteria, viruses, spore forms, etc.). In at least one embodiment, the packaging may be configured to be sealed, e.g., a water vapor-resistant packaging, optionally under an air-tight and/or vacuum seal.

Following construction of the device, it can be optionally dried, e.g., by mild desiccation, blow drying, lyophilization, or exposure to ambient air at ambient temperature, for a time sufficient for the article to be dry or at least macroscopically dry. Once the device is dry or at least macroscopically dry, it may be sealed in a container (e.g., such as an impermeable or semipermeable polymeric container) in which it can be stored and shipped to a user. In some embodiments, the LFD 100 has a shelf life of about 6 months to about 18 months. In some embodiments, the LFD 100 has a shelf life of about 6 months to about 2 years.

The instructions for use can include instructions for using the components of the kit to practice the disclosed methods of determining a risk that a subject has a disease associated with elevated CRP, BNP and/or IL6. In at least one embodiment, the instructions are present as an electronic storage data file present on a suitable computer readable storage medium, e.g., CD-ROM, diskette, flash drive, etc. In some other embodiments, the actual instructions are not present in the kit but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

In at least one embodiment, the kit includes instructions that correlate the number, intensity, or both, of visible test dots 114 with the concentration of the biomarker in the sample or with the risk of cardiometabolic disease associated with the result (for example, low risk, moderate risk, high risk). In at least one embodiment, the kit includes a reference card that correlates the number and/or intensity of visible test dots 114 with the concentrations of CRP, BNP, IL6 or a cardiometabolic disease risk biomarker 119 in the sample.

The description of the devices, porous substrates, sample pad zones 108, detection zones 110, detection agents, control nanoparticles, test dots 114, control dots 116, and other components can also be applied to the kits disclosed herein.

### EXAMPLES

### Example 1. Fabricating μPAD and Materials, Reagents and Equipment

The following non-limiting examples are illustrative of the present disclosure:

Over 30% of deaths globally are reported to be caused by cardiovascular diseases (CVDs), making CVDs recognized as one of the leading causes of death annually by the World Health Organization.¹ The high prevalence of CVD, in combination with the demographic shift towards an ageing population with high incidence of chronic diseases, make CVD a significant threat for society, necessitating accessible screening for CVD and early diagnosis of the disease.² Toward that end, the development of affordable, accurate, and timely CVD screening is critical.

Currently, screening and diagnosis of CVDs is based on subjective and objective clinical findings and serum biomarkers,^{3,4} most notably serum c-reactive protein (CRP)—an acute inflammatory protein implicated in vascular inflammation, endothelial injury, and heart disease.⁵ The compatibility of salivary CRP with serum CRP has recently been researched by several groups.^{3,6,7} Foley et al. recorded significant elevations of serum CRP (P<0.001) and salivary CRP (P<0.02) in 21 patients with CVD and noted significant associations (r=0.8) between serum and salivary CRP levels.⁸ Further, Labet et al collected saliva and plasma samples from 250 individuals with a prior history of CVD and measured salivary levels of CRP. They found a positive and significant correlation between salivary and serum CRP levels, suggesting that saliva could be an alternative means for screening and detecting CVD risk.⁹

For the detection of serum and salivary biomarkers, microfluidic paper-based analytical devices (μPADs) have been growing in popularity.^{10,11,12} Key advantages of μPADs include affordability, accessibility, and user-friendliness^{13,14}—the capability for a test to be easily used and the results interpreted by untrained personnel.¹⁵ Park et al. (2019) demonstrated the detection of CRP in a clinically relevant range using a μPAD.¹⁶ However, interpretation of the results required analysis of signal intensity, technical training and access to external devices such as a camera and image processing software. To overcome the disadvantages of signal intensity measurements, counting-based test readout methods have been developed for μPADs.^{17,18,19} Such devices can be comprised of a series of test dots 114 where the concentration of the analyte is correlated with the number of dots that exhibit a color change—removing the need for conventional interpretation of signal intensity.

In addition, an important aspect for user-friendly tests is to ensure that untrained users can safely collect and incorporate the samples onto the μPADs.¹⁵ However, painless and convenient sample collection methods, such as saliva, lead to smaller sample volumes (in the range of 1 μl-20 μl) when compared to a venous draw.²⁰ The small sample size is addressed in a μPAD with a counting-based result by involving a flow strategy that uses a running liquid. The running liquid enables sub-microliter sample volumes to flow along the entire assay.

The μPAD LFD was fabricated using a CO₂ laser cutting technique. The laser cutting technique is a high-resolution technique which allows for the fabrication of smaller device features when compared to conventional assembly methods whereby several different hydrophilic pads are cut and merged.^{22,23,24} The μPAD has a counting-based test readout to accommodate small sample volume samples and provide user-friendly testing without the need for the conventional interpretation of signal intensity.

The device consists of four zones, which are labeled in FIG. 1 as follows: running liquid zone 106, sample pad zone 108, detection zone 110 (containing test dots 114 and control dot 116, and absorption zone 112. All the zones were patterned on a single sheet of the paper material to eliminate the complexity of merging multiple pads. The detection zone exhibits a width of 1.5 mm to enable naked-eye visualization of the test results. Nitrocellulose membranes of 0.45 μm pore size were used in the fabrication of PADs for the CRP test.

Human native recombinant CRP and CRP antibodies were used for the study sample. An appropriate antibody pair was identified, which was incorporated into the device. The testing zone was impregnated with unconjugated CRP antibodies, and anti-rabbit IgG Abs (1 mg/mL) was used for the control zone. Additionally, a running liquid of de-ionized water was used for the running liquid. Given that saliva samples can vary in viscosity, the incorporation of running liquid dominates flow behavior and helps to diminish the influence of the sample fluid properties on the flow behavior. Furthermore, the assay relied upon a colorimetric change of horse radish peroxidase (HRP) conjugated to the detection antibody.

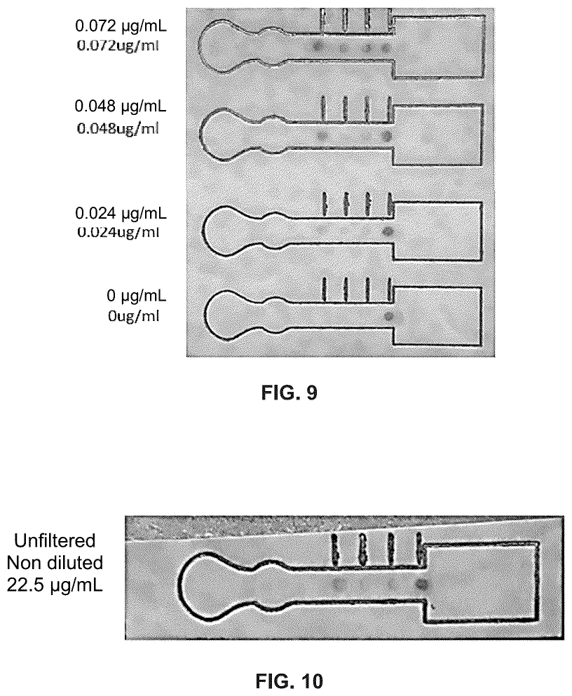

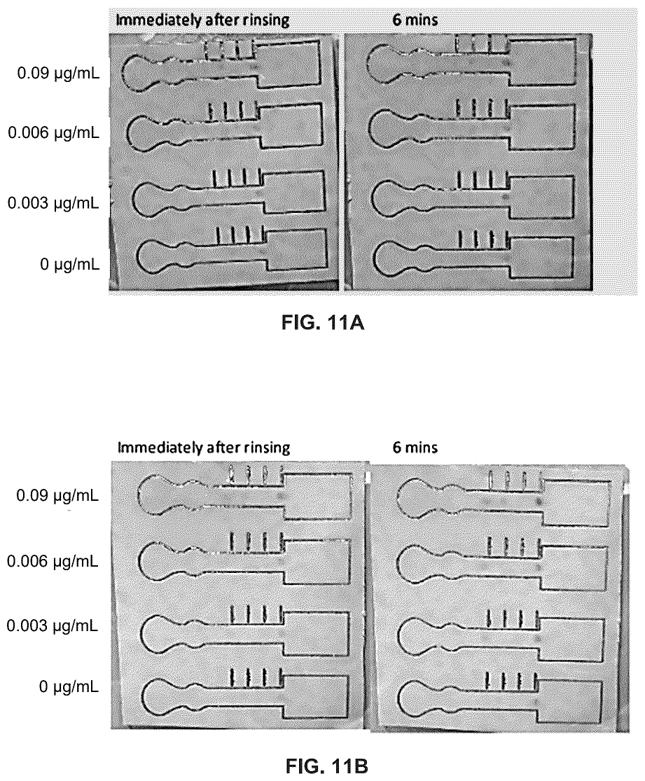

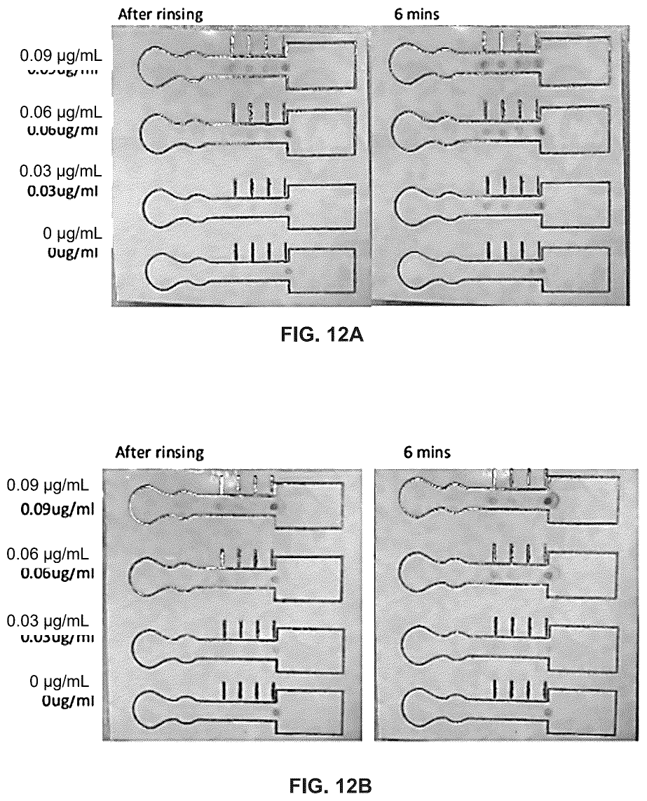

Artificial saliva samples and different concentrations of CRP were analyzed using the aforementioned counting-based μPAD LFD (FIG. 9). A human CRP test was performed to determine the total human CRP present in a sample solution prepared in a mucin-based artificial saliva spiked at different concentrations of CRP demonstrating the efficacy of the miniaturized counting based μPADs for a CRP immunoassay. After mixing, the solution was buffered to pH 8 to ensure that the final pH at the end of the test cycle does not drop below 7. In this way, exposure more closely simulates normal physiological conditions.

Each test dot was predesigned to consume a specific number of molecules of the target analyte. A volume of 0.2 μL of unconjugated anti-CRP antibody (capturing) at 1 mg/ml was spotted on each of the test dot 114 (“T1-T3”) using a repeating syringe dispenser, resulting in a maximum of 0.2 μg of antibody to be consumed at each test dot 114. As the sample flows through the counting-based μPAD, the test dot 114 will change colour when the target analyte (CRP) is consumed at the test dot 114 site. Therefore, the concentration range of CRP in the sample can be determined by counting the number of test dot 114 that change colour, providing semi-quantitative results.

Human saliva samples of heart failure patients with naturally occurring high levels of CRP were analyzed using the aforementioned counting-based μPAD LFD. To demonstrate the efficacy of the miniaturized counting-based μPADs for an CRP immunoassay, a gold standard CRP test was performed to determine the total human CRP present in a sample solution introduced to the μPAD LFD (FIG. 10).

### PROCEDURES

### 1.2 Fabricating the μPAD

In this example the μPAD was fabricated as follows:
1) A double-sided adhesive mounting layer was sandwiched between a layer of tin foil and the 0.45 μm membrane paper. Care was taken when working with the membrane as it can be fragile. Touching the membrane surface was minimized. A scraper was used carefully to flatten the sheets.
2) White printer paper was placed as a protective surface on both sides of the device and pressure was applied using a roller to ensure even bonding of the adhesive to the materials. To prevent paper from curling when conducting the test, the double-sided adhesive was applied to the underside of the device.
3) Paper material was removed with CO₂ laser cutter to provide continuous flow channel. Example settings included RGB 1000 Hz, 7 power, and 0.75 speed.
   1.3 Preparing μPAD and reagents for CRP Test

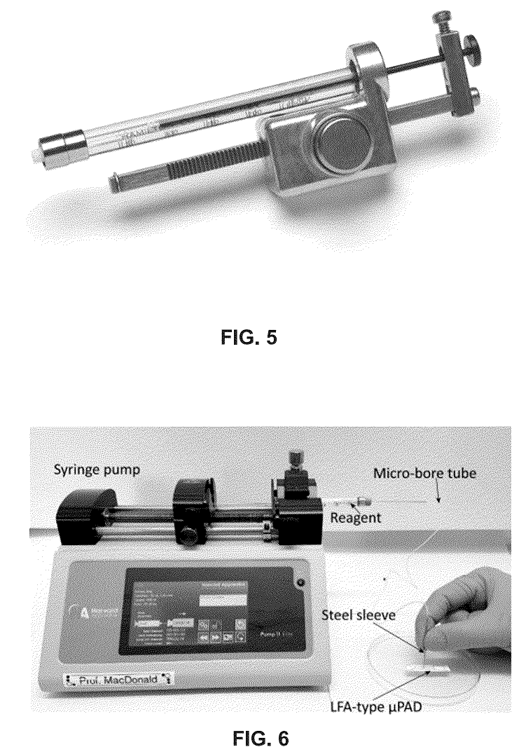

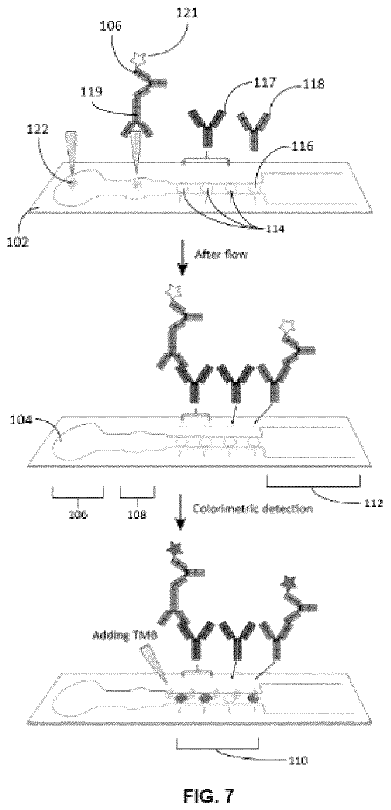

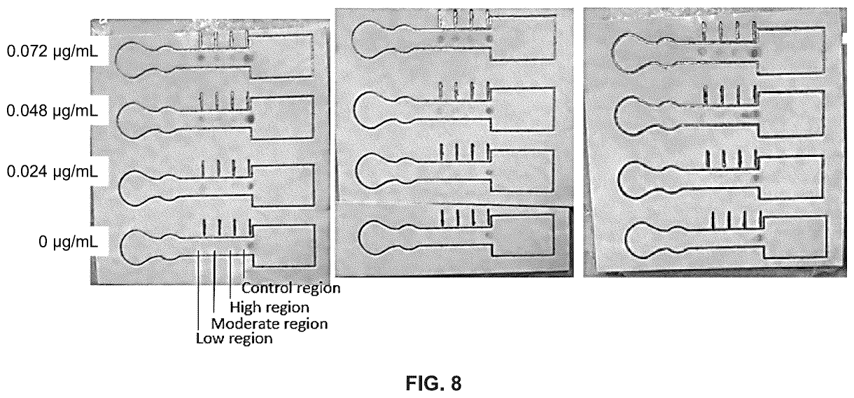

In this example, the LFDs were prepared using the following steps:
1) Antibody was diluted with PBS.
2) Serial dilutions of CRP protein were prepared in PBS.
3) Samples were mixed and dispensed.
4) Reagents were spotted onto the μPAD using a Hamilton dispenser according to FIG. 5 in 0.2 μL spots. See Table 1 for notes on products used.
5) LFDs were dried in an incubator for 30 minutes at 37° C.
6) LFDs were removed from incubator and allowed to cool for a minute.
7) LFDs were blocked with 5 w/v % skim milk for 15 minutes on a rocker.
8) LFDs were washed with 0.05 w/v % Tween-20 for 10 minutes on a rocker.
9) LFDs were dried in incubator for 90 minutes at 37° C.

### Run CRP Test

In this example, a CRP test was performed using the μPAD device as follows:
1) 3M adhesive was applied to petri dishes to gently adhere devices to the dishes without touching the LFD's membrane.
2) Test was conducted in environment to minimize evaporation of sample.
3) Within 20 seconds, 1 μL of the HRP-conjugated sample was first deposited and then the running liquid was added to the designated areas according to FIG. 4.
4) After the sample had wicked across the absorbent region of the device, 3 μL of TMB was added across the entire detection zone.
5) The LFD was very swiftly rinsed of TMB.
6) The LDFs were imaged immediately after rinsing.
7) The LFDs were imaged 2 minutes later.

### Example 2—Determination of CRP Detection Parameters

To determine the quantitative parameters of the LFD for detecting CRP in saliva, the limit of blank (LoB), limit of detection (LoD) were determined. A calibration curve was also generated to demonstrate a suitable range of CRP detection levels that can be quantified using the LFDs described herein.

### Limit of Detection

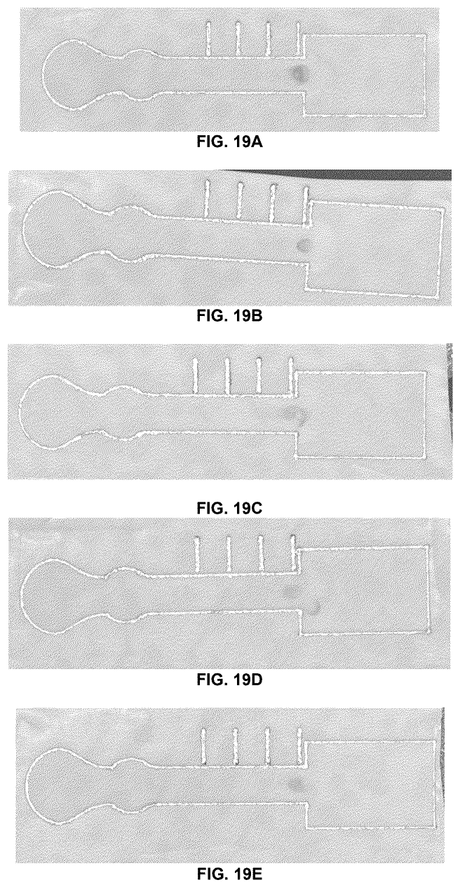

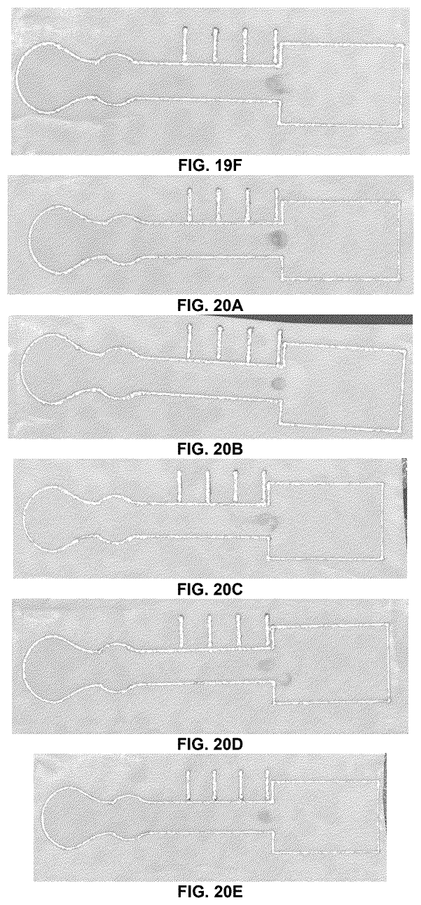

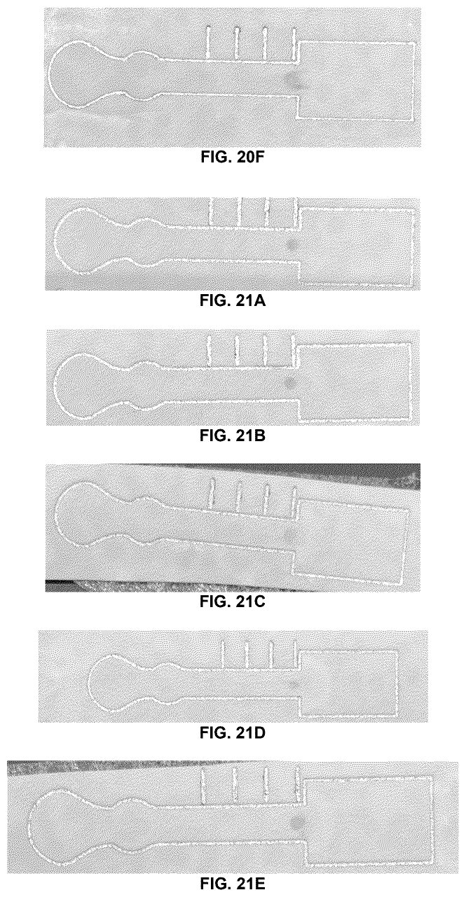

The limit of detection (LoD) for the LFD was determined by plotting a curve that correlates signal intensity to analyte concentration (FIG. 18) using a similar protocol for the CRP test as described above in Example 1. Briefly, 6 series of samples using known concentrations of 0.0005 μg/mL, 0.001 μg/mL, 0.0015 μg/mL, 0.002 μg/mL, 0.0025 μg/mL and 0.003 μg/mL were run in replicates (FIGS. 19A-F and replicates shown in FIGS. 20A-F).

The series of samples containing progressively increasing concentrations of the target analyte were applied to the device, and the corresponding signal intensities were recorded an optical reader after 3 minutes. The data points were fitted to a regression model, to establish the relationship between concentration and signal. The LoD was identified as the lowest concentration at which the measured signal exceeded the previously calculated limit of the blank (LoB) with 95% confidence.

### Limit of Blank

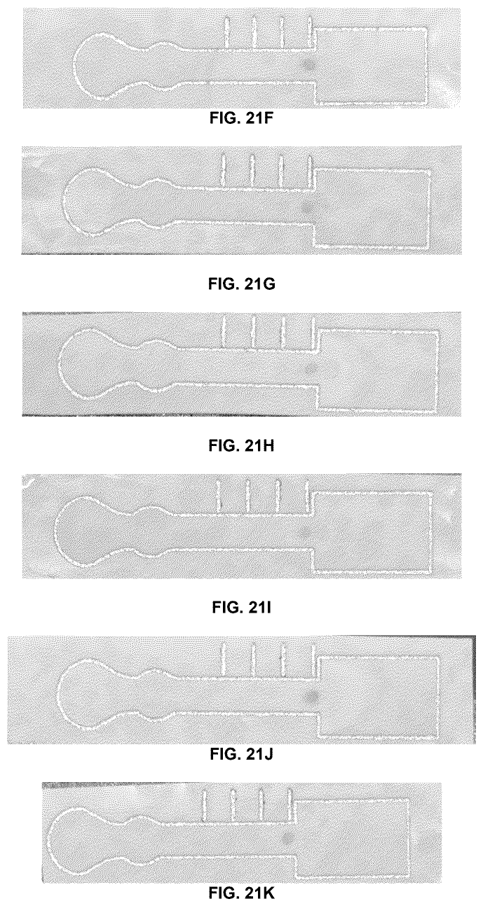

To determine the LoB samples containing no CRP were applied to LFD using the same protocol as described in Example 1. The blank samples are shown in FIGS. 21A-K, and measured in replicates (not shown) to determine a LoB of 1.815.

### Calibration Curve

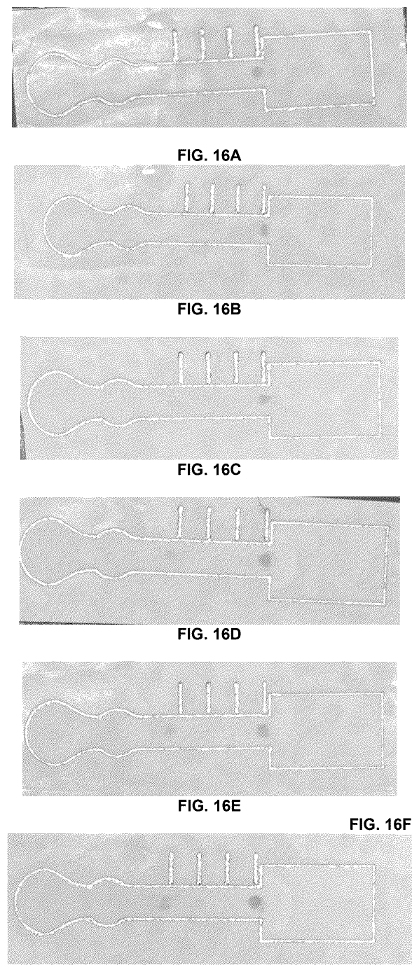

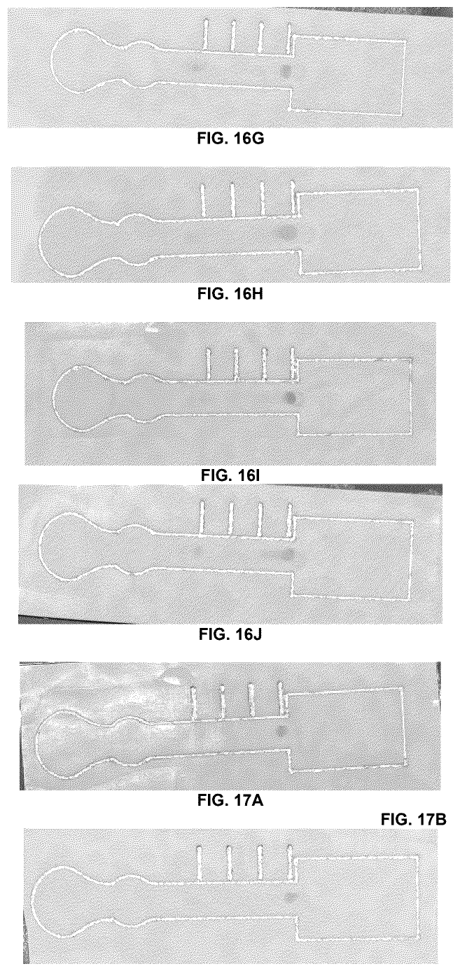

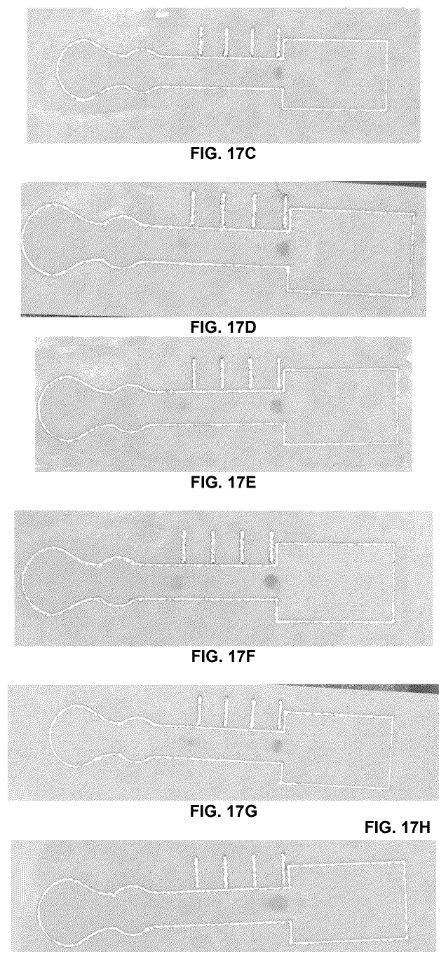

A calibration curve was generated to establish the quantitative relationship between analyte concentration and signal intensity for the LFD (FIG. 15). A series of standard solutions containing the target analyte at concentrations ranging from 0 μg/mL to 0.0072 μg/mL were prepared and applied to individual LFDs (FIGS. 16A-16J, replicates shown in FIGS. 17A-17J) using the protocol as described above in Example 1. The signal intensity was measured using an optical reader after 3 minutes. The measured values were plotted against the known analyte concentrations to produce a calibration curve. The data were fitted to a linear regression model. The resulting curve can be used to provide a reference for interpolating unknown sample concentrations and demonstrates the dynamic range and sensitivity of the device.

### Example 3—Detection of BNP Using the LFD

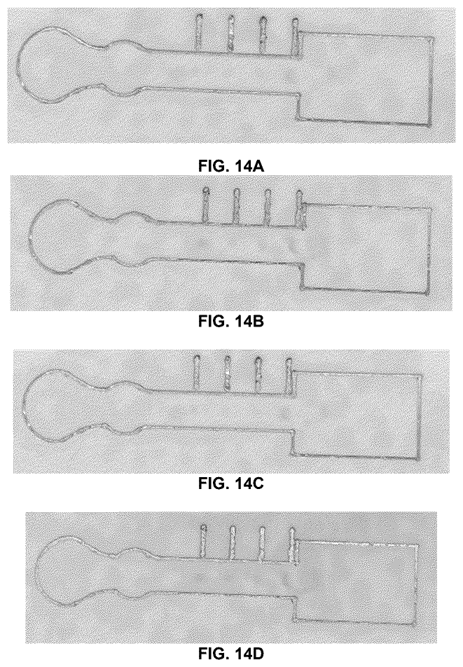

The LFD as described in Example 1 was adapted for detection of BNP. Briefly, the same test protocol as described in Example 1 was used on an LFD configured with anti-BNP antibodies to capture and detect BNP in saliva. As shown in FIGS. 14A-D, low and high levels of BNP were detected by the LFD. 5×10⁻⁷ μg/mL of BNP (low amount) was detected after a 3 minute (FIG. 14A) and a 6 minute (FIG. 14C) exposure time. 2×10⁻⁶ μg/mL of BNP (high amount) was detected after a 3 minute (FIG. 14B) and a 6 minute (FIG. 14D) exposure time.

Using the methods described above in Example 2, the LoD and a calibration curve are generated using known amounts of BNP on the LFD and the LoB is determined using blank samples containing no BNP.

### Example 4—Detection of IL6 Using the LFD

The LFD as described in Example 1 is adapted for detection of IL6. Briefly, the same test protocol as described in Example 1 is used on an LFD configured with anti-IL6 antibodies to capture and detect IL6 in saliva. 5×10⁻⁷ μg/mL of IL6 (low amount) are detected after a 3 minute and a 6 minute exposure time. 1.65×10⁻⁶ μg/mL of IL6 are detected after a 3 minute and a 6 minute exposure time.

Using the methods described above in Example 2, the LoD and a calibration curve are generated using known amounts of IL6 on the LFD and the LoB is determined using blank samples containing no IL6.

While the present application has been described with reference to what are presently considered to be some representative examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the embodiments and examples disclosed herein but should be given the broadest interpretation consistent with the description as a whole.

### Tables

TABLE 1
Materials, reagents, and equipment

| Material | Vendor, Notes |
|---|---|
| 0.45 um nitrocellulose membrane | Sigma-Aldrich, catalogue no. 1620115 |
| Goat Anti-CRP antibody (“abT”) | Sigma-Aldrich, catalogue no. SAB4701017 |
| Human CRP | Sigma Aldrich, catalogue no. SRP6267 |
| Goat HRP anti-CRP antibody | Abcam, catalogue no. ab19175 |
| (“abHRP”) | |
| Rabbit anti-goat IgG antibody | Abcam, catalogue no. ab6697 |
| (“abC”) | |
| TMB | Abcam, catalogue no. AB171522-1002 |
| Skim milk powder | For blocking, Bioshop, catalogue no. SKI400 |
| 0.05% Tween-20 in PBS | For rinsing excess blocking agent, Sigma-Aldrich, |
| | Catalogue No. P9416-100ML |
| Positional mounting adhesive | 3M, ID: 7100308642 |
| Autoclaved deionized water | For running liquid |
| Microliter delivery system | A syringe pump as pictured in FIG. 5 can be used; |
| | alternatives can include the Hamilton PB600 |
| | dispenser (dispensing 0.2 uL) to minimize loss of |
| | reagent in tubing. |

### REFERENCES

1. Benjamin E J, Benziger C P B A. Global burden of cardiovascular diseases and risk factors, 1990-2019: update from the GBD 2019 study. Journal of the American C.
2. Thomas H, Diamond J, Vieco A, Chaudhuri S, Shinnar E, Cromer S, Perel P, Mensah G A, Narula J J C et al. Global atlas of cardiovascular disease 2000-2016. Glob Heart 2018. 13, 143-163.
3. Miller, C. S.; Foley, J. D.; Floriano, P. N.; Christodoulides, N.; Ebersole, J. L.; Campbell, C. L.; Bailey, A. L.; Rose, B. G.; Kinane, D. F.; Novak M J. et al. Utility of Salivary Biomarkers for Demonstrating Acute Myocardial Infarction. J. Dent. Res. 2014, 93, 72S-79S.
4. Javaid, M. A.; Ahmed, A. S.; Durand, R.; Tran S D. Saliva as a diagnostic tool for oral and systemic diseases. J. Oral Biol. Craniofac. Res. 2016, 6, 67-76.
5. Cozlea D L, Farcas D M, Nagy A, Keresztesi A A, Tifrea R C L & C E. The impact of C reactive protein on global cardiovascular risk on patients with coronary artery disease. Curr Heal Sci J. 2013. 39, 225-231.
6. Punyadeera C. New frontiers in heart failure detection: Saliva testing. BMJ Innov. 2016, 2, 106-108.
7. Thygesen, K.; Alpert, J. S.; White H D. Universal definition of myocardial infarction. Eur. Heart J. 2007, 28, 2525-2538.
8. Foley J D, Sneed J D, Steinhubl S R, Kolasa J R, Ebersole J L, Lin Y, Kryscio R J, McDevitt J T C C & M C. Salivary biomarkers associated with myocardial necrosis: results from an alcohol septal ablation model. 2012. Oral Surg Oral Med Oral Pathol Oral Radiol 114, 616-623.
9. Labat, C.; Temmar, M.; Nagy, E.; Bean, K.; Brink, C.; Benetos, A.; Bäck M. Inflammatory mediators in saliva associated with arterial stiffness and subclinical atherosclerosis. J. Hypertens. 2013, 31, 2251-2258.
10 Yetisen, A. K., Akram, M. S. and Lowe C R. Based microfluidic point-of-care diagnostic devices. Lab on a Chip, 2013. 13 (12), pp. 2210-2251.
11 Parolo, C. and Merkoçi A. Based nanobiosensors for diagnostics. Chemical Society Reviews, 2013. 42 (2), pp. 450-457.
12. Nery E W K L. Sensing approaches on paper-based devices: a review. Analytical and bioanalytical chemistry. 2013 September; 405 (24): 7573-95.
13 Martinez A W, Phillips S T, Whitesides G M. Diagnostics for the Developing World: Microfluidic Paper-Based Analytical Devices. 2010; 82 (1): 3-10.
14. Kalish, B. and Tsutsui H. Patterned adhesive enables construction of nonplanar three-dimensional paper microfluidic circuits. Lab on a Chip, 2014. 14 (22), pp. 4354-4361.
15 Cate D M, Noblitt S D, Volckens J H C. Multiplexed paper analytical device for quantification of metals using distance-based detection. Lab on a Chip. 2015; 15 (13): 2808-18.
16. Park J P J. Pressed region integrated 3D paper-based microfluidic device that enables vertical flow multistep assays for the detection of C-reactive protein based on programmed reagent loading. Sensors and Actuators B: Chemical. 2017 Jul. 1; 246:1049-55.
17. Lewis G G, DiTucci M J P S T. Quantifying analytes in paper-based microfluidic devices without using external electronic readers. Angewandte Chemie International Edition. 2012 Dec. 14; 51 (51): 12707-10.
18 Jeong S G, Lee S H, Choi C H, Kim J L C. Toward instrument-free digital measurements: A three-dimensional microfluidic device fabricated in a single sheet of paper by double-sided printing and lamination. Lab on a Chip. 2015; 15 (4): 1188-94.
19. Yin H Y, Chu P T, Tsai W C W H. Development of a barcode-style lateral flow immunoassay for the rapid semi-quantification of gliadin in foods. Food Chemistry. 2016 Feb. 1; 192:934-42.
20 Vella S J, Beattie P, Cademartiri R, Laromaine A, Martinez A W, Phillips S T, Mirica K A W G. Measuring markers of liver function using a micropatterned paper device designed for blood from a fingerstick. Analytical chemistry. 2012 Mar. 20; 84 (6): 2883-91.
21. Fang C, Chen Z, Li L X J. Barcode lateral flow immunochromatographic strip for prostate acid phosphatase determination. Journal of pharmaceutical and biomedical analysis. 2011 Dec. 15; 56 (5): 1035-40.
22. Fung K K, Chan C P R R. Development of enzyme-based bar code-style lateral-flow assay for hydrogen peroxide determination. Analytica chimica acta. 2009 Feb. 16; 634 (1): 89-95.
23. Fung K K, Chan C P R R. Development of a creatinine enzyme-based bar-code-style lateral-flow assay. Analytical and bioanalytical chemistry. 2009 February; 393 (4): 1281-7.
24 Campbell N R, Ordunez P, Giraldo G, Morales Y A, Lombardi C, Khan T, Padwal R, Tsuyuki R T V C. WHO HEARTS: a global program to reduce cardiovascular disease burden: experience implementing in the Americas and opportunities in Canada. Canadian Journal of Cardiology. 2021 May 1; 37 (5): 744-55.
25 Li, Y., Zhong, X., Cheng, G., Zhao, C., Zhang, L., Hong, Y., Wan, Q., He, R. and Wang, Z., 2017. Hs-CRP and all-cause, cardiovascular, and cancer mortality risk: a meta-analysis. Atherosclerosis, 259, pp. 75-82.
26. Kandelouei, T., Abbasifard, M., Imani, D., Aslani, S., Razi, B., Fasihi, M., Shafiekhani, S., Mohammadi, K., Jamialahmadi, T., Reiner, Z. and Sahebkar, A., 2022. Effect of Statins on Serum level of hs-CRP and CRP in Patients with Cardiovascular Diseases: A Systematic Review and Meta-Analysis of Randomized Controlled Trials. Mediators of Inflammation, 2022 (1), p.8732360.

1. A semi-quantitative lateral flow device for measuring a cardiometabolic biomarker level in a saliva sample from a subject comprising:
I) a running liquid zone;
II) a sample pad zone configured to receive the sample;
III) a detection zone; and
IV) an absorption zone;
wherein the running liquid zone, the sample pad zone, the detection zone, and the absorption zone are coupled sequentially by a continuous flow channel, and wherein the detection zone comprises at least one test dot configured to detect the cardiometabolic biomarker in saliva and at least one control dot.

2. The LFD of claim 1, wherein the cardiometabolic biomarker comprises C-reactive protein (CRP), brain natriuretic peptide (BNP) or interleukin 6 (IL6).

3. The LFD of claim 1, wherein the detection zone comprises at least two test dots configured to detect the cardiometabolic biomarker in saliva wherein the detection zone comprises three sequential test dots configured to detect the cardiometabolic biomarker in saliva.

4. The LFD of claim 3, wherein a test dot closest to the sample pad zone is configured to detect a low amount of the cardiometabolic biomarker in the sample, a middle test dot is configured to detect a moderate amount of the cardiometabolic biomarker in the sample, and/or a test dot furthest from the sample pad zone is configured to detect a high amount of the cardiometabolic biomarker in the sample.

5. The LFD of claim 4, wherein
(a) the cardiometabolic biomarker comprises CRP, and wherein the low amount comprises 0.024 μg/mL CRP; the moderate amount comprises 0.048 μg/mL CRP; and/or the high amount comprises 0.072 μg/mL CRP;
(b) the cardiometabolic biomarker comprises BNP, and wherein the low amount comprises about 5×10⁻⁷ μg/mL or less; the moderate amount comprises at least 5×10⁻⁷ μg/mL to less than 2×10⁻⁶ μg/mL; and/or the high amount comprises about 2×10⁻⁶ μg/mL or greater; or
(c) the cardiometabolic biomarker comprises IL6, and wherein the low amount comprises about 5×10⁻⁷ μg/mL or less; the moderate amount comprises at least 5×10⁻⁷ μg/mL to less than 1.65×10⁻⁶ μg/mL; and/or the high amount comprises about 1.65×10⁻⁶ μg/mL or greater.

6. The LFD of claim 1, wherein each test dot is positioned along and coupled to the continuous flow channel and/or, wherein the sample pad zone, the detection zone, and the absorption zone and/or the running liquid zone are formed by a continuous nitrocellulose membrane.

7. The LFD of claim 6, wherein the nitrocellulose membrane has a pore size of 0.45 μm, and/or the nitrocellulose membrane is coupled to a support layer by an adhesive layer, optionally the support layer comprises aluminum foil.

8. The LFD of claim 1, wherein:
(a) the at least one test dot comprises a capture agent, optionally the capture agent comprises an anti-CRP antibody, an anti-BNP antibody or an anti-IL6 antibody, further optionally each of the at least one test dot comprises 0.2 μg of anti-CRP antibody, anti-BNP antibody or anti-IL6 antibody; and/or
(b) the at least one control dot comprises a control capture agent.

9. The LFD of claim 1, wherein the device further measures one or more other cardiometabolic disease risk biomarkers, optionally the one or more other biomarkers of cardiometabolic disease comprises NT-proBNP.

10. The LFD of claim 1, wherein the device is adapted to determine the risk that the subject has a cardiometabolic disease, optionally the cardiometabolic disease comprises any combination of acute coronary syndrome, acute heart failure, myocardial infarction, stable atherosclerotic plaques, unstable angina, systemic hypertension, and cardiovascular mortality.

11. The LFD of claim 1, wherein the test dot has a width of about 1.5 mm and/or the device is for use in a colorimetric assay, optionally the colorimetric assay is an immunoassay.

12. A method of manufacturing the LFD of claim 1, comprising:
I) mounting a nitrocellulose membrane to a support layer with an adhesive layer to form a body of the device;
II) cutting the nitrocellulose membrane with a laser cutter to provide a continuous flow channel, wherein the continuous flow channel comprises a running liquid zone, a sample pad zone, a detection zone, and an absorption zone;
III) adding at least one cardiometabolic biomarker capture agent to at least one test dot in the detection zone and adding at least one control capture agent to at least one control dot in the detection zone; and
IV blocking the nitrocellulose membrane with a blocking agent.

13. The method of claim 12, wherein the method further comprises:
V) washing the nitrocellulose membrane with a washing solution, optionally the washing solution comprises Tween 20.

14. The method of claim 12, wherein the blocking agent comprises skim milk.

15. A method of determining a risk that a subject has a cardiometabolic disease, comprising:
I) combining a saliva sample from the subject with a cardiometabolic biomarker detection agent to form a test sample;
II) applying the test sample to a sample pad zone from an LFD as defined in claim 1;
III) applying a running liquid to a running liquid zone of the LFD in II); and
IV) applying a visualization agent to the detection zone;
wherein the cardiometabolic biomarker detection agent and the visualization agent result in a visual change when combined, and wherein the visual change is indicative of the risk that the subject has cardiovascular disease.

16. The method of claim 15, wherein the cardiometabolic biomarker detection agent comprises a C-reactive protein (CRP) detection agent, a brain natriuretic peptide (BNP) detection agent or a interleukin 6 (IL6) detection agent, and/or the cardiometabolic biomarker detection agent is an anti-CRP antibody conjugated to horseradish peroxidase, anti-BNP antibody conjugated to horseradish peroxidase or anti-IL6 antibody conjugated to horseradish peroxidase.

17. The method of claim 15, wherein (a) the visualization agent comprises 3,3′,5,5′-Tetramethylbenzidine (TMB), (b) the cardiometabolic biomarker detection agent binds a different cardiometabolic biomarker epitope than the cardiometabolic biomarker capture agent in the LFD test dots, and/or (c) the running liquid comprises deionized water.

18. The method of any one of claim 15, wherein a low amount of the cardiometabolic biomarker in the saliva is indicative that the subject has a low risk of cardiovascular disease; a moderate amount of the cardiometabolic biomarker in the saliva is indicative that the subject has a moderate risk of cardiovascular disease; or a high amount the cardiometabolic biomarker in the saliva is indicative that the subject has a moderate risk of cardiovascular disease.

19. The method of claim 18, wherein:
(a) the cardiometabolic biomarker comprises CRP, and wherein the low amount comprises 0.024 μg/mL CRP; the moderate amount comprises 0.048 μg/mL CRP; and/or the high amount comprises 0.072 μg/mL CRP;
(b) the cardiometabolic biomarker comprises BNP, and wherein the low amount comprises about 5×10⁻⁷ μg/mL or less; the moderate amount comprises at least 5×10⁻⁷ μg/mL to less than 2×10⁻⁶ μg/mL; and/or the high amount comprises about 2×10⁻⁶ μg/mL or greater; or
(c) the cardiometabolic biomarker comprises IL6, and wherein the low amount comprises about 5×10⁻⁷ μg/mL or less; the moderate amount comprises at least 5×10⁻⁷ μg/mL to less than 1.65×10⁻⁶ μg/mL; and/or the high amount comprises about 1.65×10⁻⁶ μg/mL or greater.

20. A kit comprising the device of claim 1, a running liquid, a solution comprising a colorimetric cardiometabolic biomarker detection agent, and a solution comprising a colorimetric visualization agent, optionally further comprising at least one receptable, at least one dropper, and/or instructions for use.
